(19) United States
(12) Patent Application Publication
(10) Pub. No.: US 2003/0141260 A1
(43) Pub. Date: Jul. 31, 2003

(54) Oxygen-enhanced pathogen inactivation

(76) Inventors: Frank Corbin III, Littleton, CO (US); Dennis Hlavinka, Arvada, CO (US); Laura Goodrich, Lakewood, CO (US); Raymond P. Goodrich, Lakewood, CO (US)

Correspondence Address: GREENLEE WINNER AND SULLIVAN P C, 5370 MANHATTAN CIRCLE, SUITE 201, BOULDER, CO, 80303, US

(21) Appl. No.: 10/328,717

(22) Filed: Dec. 23, 2002

Related U.S. Application Data
(60) Provisional application No. 60/344,109, filed on Dec. 28, 2001.

(51) Int. Cl. A61L 2/08

(52) U.S. Cl. 210/748; 210/758; 210/764; 422/22; 422/29

(57) ABSTRACT

Methods for decontaminating fluids such as blood products, particularly platelets or red blood cells, are provided. The methods include mixing substantially non-toxic amounts of an endogenous photosensitizer such as riboflavin, or an endogenously-based derivative photosensitizer, with the fluid, increasing the dissolved oxygen content of the fluid to an amount sufficient to enhance a reaction of the photosensitizer in which singlet oxygen is formed and reduce competing reactions; and exposing the fluid to visible light photoradiation to activate the photosensitizer and substantially inactivate the pathogens. Blood products produced by the method, and systems for carrying out the method are also provided.

### CROSS-REFERENCE TO RELATED APPLICATIONS

This application claims priority to U.S. Provisional Application No. 60/344,109 filed Dec. 28, 2001, which is incorporated herein by reference to the extent not inconsistent herewith.

### BACKGROUND

Contamination of blood supplies with infectious microorganisms such as HIV, hepatitis and other viruses and bacteria presents a serious health hazard for those who must receive transfusions of whole blood or administration of various blood components such as platelets, red cells, albumin, and other components isolated from blood. Blood screening procedures may miss contaminants, and sterilization procedures may damage the biological product being sterilized. Efficient, effective methods are needed for decontaminating biological products without damaging them.

Solvent detergent methods of blood component decontamination work by dissolving phospholipid membranes surrounding viruses such as HIV, and do not damage protein components of blood; however, if blood cells are present, such methods cannot be used because of damage to cell membranes.

The use of photosensitizers, compounds which absorb light of a defined wavelength and transfer the absorbed energy to an energy acceptor, has been proposed for blood component sterilization. For example, European Patent application 196,515 published Oct. 8, 1986, suggests the use of non-endogenous photosensitizers such as porphyrins, psoralens, acridine, toluidines, flavine (acriflavine hydrochloride), phenothiazine derivatives, and dyes such as neutral red and methylene blue, as blood additives. Protoporphyrin, which occurs naturally within the body, can be metabolized to form a photosensitizer; however, its usefulness is limited in that it degrades desired biological activities of proteins. Chlorpromazine, is also exemplified as one such photosensitizer; however its usefulness is limited by the fact that it should be removed from any fluid administered to a patient after the decontamination procedure because it has a sedative effect.

Goodrich, R. P., et al. (1997), “The Design and Development of Selective, Photoactivated Drugs for Sterilization of Blood Products,” Drugs of the Future 22:159-171 provides a review of some photosensitizers including psoralens, and some of the issues of importance in choosing photosensitizers for decontamination of blood products. The use of texaphyrins for DNA photocleavage is described in U.S. Pat. No. 5,607,924 issued Mar. 4, 1997 and U.S. Pat. No. 5,714,328 issued Feb. 3, 1998 to Magda et al. The use of sapphyrins for viral deactivation is described in U.S. Pat. No. 5,041,078 issued Aug. 20, 1991 to Matthews, et al. Inactivation of extracellular enveloped viruses in blood and blood components by Phenthiazin-5-ium dyes plus light is described in U.S. Pat. No. 5,545,516 issued Aug. 13, 1996 to Wagner. The use of porphyrins, hematoporphyrins, and merocyanine dyes as photosensitizing agents for eradicating infectious contaminants such as viruses and protozoa from body tissues such as body fluids is disclosed in U.S. Pat. No. 4,915,683 issued Apr. 10, 1990 and related U.S. Pat. No. 5,304,113 issued Apr. 19, 1994 to Sieber et al. The mechanism of action of such photosensitizers is described as involving preferential binding to domains in lipid bilayers, e.g. on enveloped viruses and some virus-infected cells. Photoexcitation of membrane-bound agent molecules leads to the formation of reactive oxygen species such as singlet oxygen. U.S. Pat. No. 4,727,027 issued Feb. 23, 1988 to Wiesehahn, G. P., et al. discloses the use of furocoumarins including psoralen and derivatives for decontamination of blood and blood products, but teaches that steps must be taken to reduce the availability of dissolved oxygen and other reactive species in order to inhibit denaturation of biologically active proteins.

Photoinactivation of viral and bacterial blood contaminants using halogenated coumarins is described in U.S. Pat. No. 5,516,629 issued May 14, 1996 to Park, and related U.S. Pat. No. 6,251,644 to Sowemimo-Coker et al. issued Jun. 26, 2001. U.S. Pat. No. 5,587,490 issued Dec. 24, 1996 to Goodrich Jr., R. P., et al. and U.S. Pat. No. 5,418,130 to Platz, et al. disclose the use of substituted psoralens for inactivation of viral and bacterial blood contaminants. The latter patent also teaches the necessity of controlling free radical damage to other blood components. U.S. Pat. No. 5,654,443 issued Aug. 5, 1997 to Wollowitz et al. teaches new psoralen compositions used for photodecontamination of blood. U.S. Pat. No. 5,709,991 issued Jan. 20, 1998 to Lin et al. teaches the use of psoralen for photodecontamination of platelet preparations and removal of psoralen afterward. U.S. Pat. No. 5,360,734 issued Nov. 1, 1994 to Chapman et al. addresses the problem of prevention of damage to other blood components. U.S. Pat. No. 5,120,649 issued Jun. 9, 1992, related U.S. Pat. No. 5,232,844 issued Aug. 3, 1993 to Horowitz, et al., related U.S. Pat. No. 5,658,722 issued Aug. 19, 1997 to Margolis-Nunno et al., related U.S. Pat. No. 5,858,643 issued Jan. 12, 1999 to Ben Hur et al., related U.S. Pat. No. 5,981,163 issued Nov. 9, 1999, to Horowitz et al. related U.S. Pat. No. 6,077,659 issued Jun. 20, 2000, to Ben Hur et al. related U.S. Pat. No. 6,214,534 issued Apr. 10, 2001, to Horowitz et al. on and related U.S. Pat. No. 6,294,361 issued Sep. 25, 2001 to Horowitz et al. also disclose the need for the use of “quenchers” in combination with photosensitizers which attack lipid membranes. U.S. Pat. Nos. 5,232,844 and 6,294,361 state that the process may be carried out in the presence of an oxidizer, which can be oxygen, and that the concentration of oxygen can be the endogenous quantity, or can be modified by placement of the material being treated in an atmosphere designed to lower or raise oxygen concentration. However, the examples of these patents teach benefits of lowering oxygen content, and of using normal aeration combined with quencher (compared with using a nitrogen atmosphere), thereby effectively teaching against using an increased oxygen concentration. U.S. Pat. No. 5,981,163 teaches benefits of deoxygenization. U.S. Pat. Nos. 6,077,659 and 5,858,643 disclose using vitamin E or derivatives thereof to prevent potassium ion leakage from red blood cells after irradiation with porphyrin-like photosensitizers. U.S. Pat. No. 4,386,069 issued May 31, 1983 to Estep discloses an additive solution to enhance preservation of normal red cell morphology during storage comprising a fatty ester which includes at least two ester linkages comprising fatty hydrocarbon groups of about four to twelve carbon atoms each.

Photosensitizers which attack nucleic acids are known to the art. U.S. Pat. No. 5,342,752 issued Aug. 30, 1994 to Platz et al. discloses the use of compounds based on acridine dyes to reduce parasitic contamination in blood matter comprising red blood cells, platelets, and blood plasma protein fractions. These materials, although of fairly low toxicity, do have some toxicity e.g. to red blood cells. U.S. Pat. No. 5,798,238 to Goodrich, Jr., et al., discloses the use of quinolone and quinolone compounds for inactivation of viral and bacterial contaminants.

Binding of DNA with photoactive agents has been exploited in processes to reduce lymphocytic populations in blood as taught in U.S. Pat. No. 4,612,007 issued Sep. 16, 1986 and related U.S. Pat. No. 4,683,889 issued Aug. 4, 1987 to Edelson.

Riboflavin (7,8-dimethyl-10-ribityl isoalloxazine) has been reported to attack nucleic acids. Photoalteration of nucleic acid in the presence of riboflavin is discussed in Tsugita, A, et al. (1965), “Photosensitized inactivation of ribonucleic acids in the presence of riboflavin,” Biochimica et Biophysica Acta 103:360-363; and Speck, W. T. et al. (1976), “Further Observations on the Photooxidation of DNA in the Presence of Riboflavin,” Biochimica et Biophysica Acta 435:39-44. Binding of lumiflavin (7,8,10-trimethylisoalloxazine) to DNA is discussed in Kuratomi, K., et al. (1977), “Studies on the Interactions between DNA and Flavins,” Biochimica et Biophysica Acta 476:207-217. Hoffmann, M. E., et al. (1979), “DNA Strand Breaks in Mammalian Cells Exposed to Light in the Presence of Riboflavin and Tryptophan,” Photochemistry and Photobiology 29:299-303 describes the use of riboflavin and tryptophan to induce breaks in DNA of mammalian cells after exposure to visible fluorescent light or near-ultraviolet light. The article states that these effects did not occur if either riboflavin or tryptophan was omitted from the medium. DNA strand breaks upon exposure to proflavine and light are reported in Piette, J. et al. (1979), “Production of Breaks in Single- and Double-Stranded Forms of Bacteriophage ΦX174 DNA by Proflavine and Light Treatment,” Photochemistry and Photobiology 30:369-378, and alteration of guanine residues during proflavine-mediated photosensitization of DNA is discussed in Piette, J., et al. (1981), “Alteration of Guanine Residues during Proflavine Mediated Photosensitization of DNA,” Photochemistry and Photobiology 33:325-333.

J. Cadet, et al. (1983), “Mechanisms and Products of Photosensitized Degradation of Nucleic Acids and Related Model Compounds,” Israel J. Chem. 23:420-429, discusses the mechanism of action by production of singlet oxygen of rose bengal, methylene blue, thionine and other dyes, compared with mechanisms not involving production of singlet oxygen by which nucleic acid attack by flavin or pteron derivatives proceeds. Riboflavin is exemplified in this disclosure as having the ability to degrade nucleic acids. Korycka-Dahl, M., et al. (1980), “Photodegradation of DNA with Fluorescent Light in the Presence of Riboflavin, and Photoprotection by Flavin Triplet-State Quenchers,” Biochimica et Biophysica Acta 610:229-234 also teaches that active oxygen species are not directly involved in DNA scission by riboflavin. Peak, J. G., et al. (1984), “DNA Breakage Caused by 334-nm Ultraviolet Light is Enhanced by Naturally Occurring Nucleic Acid Components and Nucleotide Coenzymes,” Photochemistry and Photobiology 39:713-716 further explores the mechanism of action of riboflavin and other photosensitizers. However, no suggestion is made that such photosensitizers be used for decontamination of medical fluids. Korycka-Dahl, M. and Richardson, T. (1980), “Photodegradation of DNA with Fluorescent Light in the Presence of Riboflavin, and Photoprotection by Flavin Triplet-State Quenchers,” Biochimica et Biophysica Acta 610:229-234, discusses the formation of superoxide anions generated upon illumination of nucleic acid in solution with riboflavin. Certain quenchers protected DNA from photodegradation, but alpha-tocopherol did not have a protective effect. The article concluded that active oxygen species are not involved in DNA photodegradation using riboflavin.

Sterilization procedures which do not damage cellular blood components but effectively inactivate infectious viruses and other microorganisms and contaminants are disclosed in U.S. Pat. Nos. 6,258,577, 6,277,337, 6,268,120 and PCT publications WO 01/28599, WO 00/04930, WO 02/26270, WO 02/43485, WO 02/32469, WO 01/96340, WO 01/94349, WO 01/28599, WO 01/23413, and U.S. patent application Ser. Nos. 09/725,426, 09/586,147, 09/777,727, 09/677,375, 09/596,429, 10/247,262, and 10/159,781.

Apparatuses for decontamination of blood have been described in U.S. Pat. No. 5,290,221 issued Mar. 1, 1994 to Wolfe, Jr., et al. and U.S. Pat. No. 5,536,238 issued Jul. 16, 1996 to Bischof. U.S. Pat. No. 5,290,221 discloses the irradiation of fluid in a relatively narrow, arcuate gap. U.S. Pat. No. 5,536,238 discloses devices utilizing optical fibers extending into a filtration medium. Both patents recommend as photosensitizers benzoporphyrin derivatives which have an affinity for cell walls.

Blood separation devices are disclosed, e.g. in PCT publication WO 99/11305 and WO 01/66172.

All publications and patent applications referred to herein are hereby incorporated by reference to the extent not inconsistent herewith.

### SUMMARY

Although prior publications indicate a belief in the art that excess oxygen would be detrimental to decontamination systems involving pathogen kill by means of photoactive molecules, it has been discovered, surprisingly, that when 7,8-demethyl-10 ribityl isoalloxazine (riboflavin) was used as the photoactivator, the dissolved oxygen in the fluid being decontaminated dropped substantially. Oxygen was being consumed. Addition of oxygen to the system, either by vigorous mixing of the fluid with air, or by providing an oxygen-enriched atmosphere in contact with the fluid, optionally also with mixing to increase dissolved oxygen in the fluid, increased pathogen kill without undue damage to desired biological components in the system.

Blood products are preferred fluids for decontamination by the processes of this invention, including whole blood, platelets, and red blood cells. Platelets and red blood cells are preferred products, with platelets being most preferred. Adding oxygen to the fluid speeded up the decontamination process and resulted in healthier platelets. When the photoactivation method is used in the absence of oxygen or air pathogen inactivation stops after a short initial burst. Adding air improves pathogen inactivation, agitating the fluid to increase the amount of air dissolved in the fluid further improves pathogen inactivation, and adding pure oxygen dramatically improves pathogen inactivation. However, the use of oxygen alone, in the absence of a photoactivator, does not substantially inactivate pathogens.

This invention provides methods for treating a fluid to inactivate microorganisms which may be present therein. A method of this invention comprises:

(a) mixing an inactivation-effective, substantially non-toxic amount of an endogenous photosensitizer or endogenously-based derivative photosensitizer with said fluid;

(b) increasing the dissolved oxygen content of said fluid to an amount sufficient to enhance reaction of the photosensitizer in which singlet oxygen and reactive oxygen species (ROS) are formed and preferably reduce competing reactions;

(c) exposing said fluid to photoradiation, preferably visible light radiation, of sufficient energy to activate the photosensitizer, for a sufficient time to substantially inactivate said microorganisms.

Microorganisms are completely inactivated, also referred to herein as “neutralized,” i.e. rendered unable to reproduce, or are substantially inactivated, which means the fluid is decontaminated to a level sufficient to meet requirements for intravenous introduction into a human body.

This invention also provides systems for performing the decontamination methods, including compositions useful in such systems.

### BRIEF DESCRIPTION OF THE DRAWINGS

FIG. 1 is a graph showing inactivation of bovine viral diarrhea virus (BVDV) in 27% platelets in plasma in the presence of riboflavin with and without oxygen.

FIG. 2 is a Jablonski diagram showing possible photochemical reactions of (7,8-dimethyl-10-ribityl isoalloxazine) riboflavin and related compounds in protein-containing solutions, catalyzed by photoradiation.

FIG. 3 is a graph showing inactivation of bovine viral diarrhea virus (BVDV) using isoalloxazine (riboflavin) as a photosensitizer, with air, as a function of the energy of light radiation applied.

FIG. 4 is a graph showing inactivation of BVDV using 7,8-dimethyl-10-ribityl isoalloxazine (riboflavin) as a photosensitizer using air. The graph compares the efficiency of the process when the light source is filtered with mylar or unfiltered.

FIG. 5 is a graph showing inactivation of BVDV (5% spike) at 50 micromolar riboflavin at light flux 2.5 J/cm²/min (no mylar placed over the light banks), and 1.85 J/cm²/min (one sheet of mylar placed over the light banks) at mixing speeds of 135 and 149 cpm.

FIG. 6 is a graph showing pseudorabies (PSR) virus inactivation as a function of energy (J/cm²/min) using 50 μm riboflavin, mixed with air at 149 cpm at 27:73 and 33:67 percent platelets (in plasma) to percent storage solution at flux rates of both 1.24 and 1.14 J/cm²/min achieved by placing two mylar sheets (for 1.24 J/cm²/min) or three mylar sheets for 1.14 J/cm²/min over the light banks.

FIG. 7 is a graph showing PSR virus inactivation as a function of time (minutes) using 50 micromolar riboflavin, mixed with air at 149 cpm at 27:73 and 33:67 percent platelets (in plasma) to percent storage solution at both 1.24 and 1.14 J/cm²/min achieved by placing two mylar sheets (for 1.24 J/cm²/min) or three mylar sheets for 1.14 J/cm²/min over the light banks.

FIG. 8 is a graph showing PSR virus inactivation as a function of time (minutes) using 50 micromolar riboflavin, mixed with air at 149 cpm at both 1.5 and 2.0 J/cm²/min achieved by placing mylar sheets over the light banks, with and without vitamin E.

FIG. 9 is a graph showing PSR virus inactivation as a function of energy (J/cm²/min) using 50 micromolar riboflavin, mixed with air at 149 cpm at both 1.5 and 2.0 J/cm²/min achieved by placing mylar sheets over the light banks, with and without vitamin E.

FIG. 10 is a graph showing BVDV inactivation as a function of time (minutes) using 50 micromolar riboflavin, mixed with air at 149 cpm at both 1.5 and 2.0 J/cm²/min achieved by placing mylar sheets over the light banks, with and without vitamin E.

FIG. 11A is a perspective view of a blood collection and apheresis apparatus used in this invention with associated collection and photosensitizer bags. FIG. 11B is an enlarged view of the collection and photosensitizer bags shown in FIG. 11A.

FIG. 12A depicts a pathogen eradication treatment station. FIG. 12B is an enlarged view of the bar code depicted on the bags of FIG. 12A. FIG. 12C is an enlarged view of the bag sealing accessory depicted in FIG. 12A.

### DETAILED DESCRIPTION

Fluids decontaminated using methods of this invention may be any fluids likely to be contaminated with microorganisms, preferably fluids comprising living cells and/or biologically-active protein. A preferred fluid for decontamination using methods of this invention is a fluid comprising one or more components selected from the group consisting of protein, blood and blood constituents, e.g. platelets, red cells, plasma, and plasma protein such as albumin. Platelets and red blood cells are preferred components. As is known to the art, collected platelets comprise a large proportion of carried over plasma, e.g. 250 ml of collected platelets usually contains about 3.0×10¹¹ or ×10¹² platelets and the balance plasma. As used herein, the term “platelets” refers to platelets in plasma as collected, with carried-over plasma (not concentrated). Platelets, preferably at a ratio of between about 20:80 to about 90:10 platelets (in plasma):storage solution), or between about 20:80 and about 35:65 platlets (in plasma):storage solution, are most preferred components of the fluid to be decontaminated.

Preferred photosensitizers (also referred to herein as “photoactivators”) are endogenous alloxazines, K vitamins and vitamin L, specifically 7,8-dimethyl-10-ribityl isoalloxazine, (riboflavin) 7,8-dimethylalloxazine, 7,8,10-trimethylisoalloxazine, alloxazine mononucleotide, isoalloxazine-adenosine dinucleotide, and isoalloxazine derivatives and analogs as set forth in U.S. Pat. No. 6,268,120 and U.S. patent application Ser. No. 09/777,727, both of which are incorporated herein by reference to the extent not inconsistent herewith. Specifically, the terms “endogenously-based photosensitizers” and “isoalloxazine derivative photosensitizers” are synonymous and mean compounds having the structure:

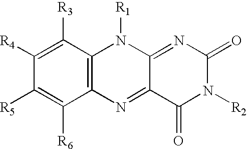

wherein R₁, R₂, R₃, R₄, R₅ and R₆ are, independently from one another, selected from the group consisting of hydrogen, optionally substituted hydrocarbyl, alcohol, amine, polyamine, sulfate, phosphate, halogen selected from the group consisting of chlorine, bromine and iodine, salts of the foregoing, and —NR^{a}—(CR^{b}R^{c})ₙ—X wherein X is a halogen selected from the group consisting of chlorine, bromine and iodine, R^{a}, R^{b} and R^{c} are, independently of each other, selected from the group consisting of hydrogen, optionally substituted hydrocarbyl, and halogen selected from the group consisting of chlorine, bromine and iodine, and n is an integer from 0 to 20;

provided that R₁ is not —OH or a straight chain alkyl group where the second carbon of the chain is substituted with —OH or ═O; and R₁ is not a 2-, 3-, 4- or 5-carbon straight chain alkyl that terminates in —OH, —C.H., or —H when R₂, R₃ and R₆ are H, and R₄ and R₅ are CH₃; R₁ is not —CH₂CH₂—(CHOH)₂—CH₃ or —CH₂CH₂—(CHOH)₂—CH₂SO₄ or 1′-D-sorbityl or 1′-D-dulcityl or 1′-D-rhamnityl or 1′-D,L-glyceryl or —CH₂—O—C(O)—CH₃ or —CH₂—O—C(O)—CH₂CH₃ or 2′, 3′, 4′, 5′-di-O-isopropyridene-riboflavin or 8-aminooctyl when R₂, R₃ and R₆ are H and R₄ and R₅ are CH₃; R₁ is not 1′-D-sorbityl or 1′-D-dulcityl when R₄ and R₅ are both chlorines and when R₂, R₃ and R₆ are all hydrogens; R₅ is not ethyl or chloro when R₁ and R₄ are methyl and R₂, R₃ and R₆ are all hydrogens; R₄ and R₅ are not both methoxy or both tetramethylene when R₁ is methyl and R₂, R₃ and R₆ are all hydrogens; R₂ is not —CH₂CH₂NH when R₁, R₄ and R₅ are CH₃ and R₃ and R₆ are H; R₂ is not

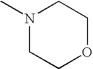

when R₁, R₄ and R₅ are CH₃ and R₃ and R₆ are H; R₅ is not chloro when R₄ is methoxy and R₁ is ethyl-2′N-pyrrolidino and R₂, R₃, and R₆ are hydrogen; R₁ is not N,N-dimethylaminopropyl or N,N-diethylaminoethyl when R₅ is chloro or methyl and R₂, R₃, R₄ and R₆ are hydrogen; R₃ is not —NH(CH₂CH₂)Cl when R₆ is —NH₂ and R₁, R₂, R₄ and R₅ are H; R₁, R₄, R₅ are not all methyl groups when all of R₂, R₃ and R₆ are hydrogens; R₁, R₄, R₅ and R₂ are not all methyl groups when R₃ and R₆ are hydrogens; R₂ is not carboxymethyl when R₁, R₄ and R₅ are methyl and R₃ and R₆ are hydrogen; R₄ is not —NH₂ when R₁ and R₅ are methyl and R₂, R₃ and R₆ are all hydrogen; R₁ is not a phenyl group when R₄ and R₅ are methyl and R₂, R₃ and R₆ are all H; R₁ is not methyl or N,N-dimethylaminoethyl when all of R₂, R₃, R₄, R₅ and R₆ are hydrogen; R₂, R₄, R₅ are not all methyl when R₁ is acetoxyethyl and R₃ and R₆ are hydrogen; R₅ is not methyl when R₁ is N,N-diethylaminoethyl and R₂, R₃, R₄ and R₆ are all hydrogen; R₄ and R₅ are not both chlorine when R₁ is methyl and R₂, R₃ and R₆ are all hydrogen; R₁ is not ethyl, β-chloroethyl, n-butyl, anilino, benzyl, phenyl, p-tolyl or p-anisyl when R₅ is NH₂ and R₂, R₃, R₄ and R₆ are all hydrogen; and R₄ is not chlorine when R₁ is N,N-dimethylaminopropyl and R₂, R₃, R₅ and R₆ are all hydrogen.

In one group of compounds, n is an integer between 0 and 5. In another group of compounds, n is an integer from 0 to 10. In another group of compounds, n is an integer from 0 to 20.

Compounds containing any combination of substituents or members of the Markush groups specified above are within the scope of the invention. All compounds of the invention have the ability to neutralize microorganisms. All substituents of the compounds of the invention may be the same, all substituents may be different, or any combination of substituents may be the same or different. Substituents with a specified function, for example those that impart water solubility to the compound, may be included at any of R₁₋₂₆. Compounds of the invention include all those compounds with the isoalloxazine backbone (shown below):

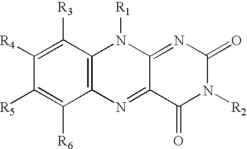

where R₁-R₆ are substituted with various substituents, as described elsewhere, except those previously known to the art. The substituents included in the compounds and used in the methods of the invention may be any substituent not having structures or reactivity which would substantially interfere with the desired microorganism neutralization of the microorganism neutralizer, as may readily be determined without undue experimentation by those skilled in the art.

The invention provides a class of compounds wherein a plurality of R₁, R₂, R₃, R₄, R₅ and R₆ are neither CH₃ nor H; and a class of compounds wherein one of R₁, R₂, R₃, R₄, R₅ and R₆ is neither CH₃ nor H. Particular embodiments of compounds of those classes include those wherein a R₁, R₂, R₃, R₄, R₅ or R₆ which is neither CH₃ nor H imparts substantial water solubility to the microorganism neutralizer. Preferred examples of these compounds are:

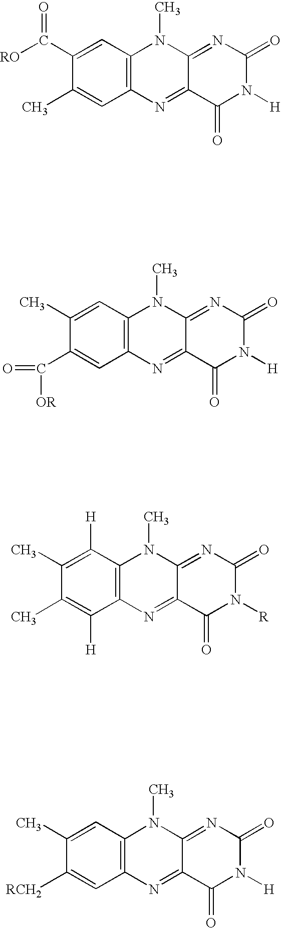

wherein R is a substituent imparting water solubility to the molecule, including, but not limited to, ascorbate, alcohol, polyalcohol; amine or polyamines, straight chain or cyclic saccharides, sulfates, phosphates, alkyl chains optionally substituted with —OH at any position, glycols, including polyethylene glycol and polyethers.

Another class of compounds of the invention include those wherein a R₁, R₂, R₃, R₄, R₅ or R₆ that is neither H nor CH₃ contains a halogen or is a halogen, wherein the halogen is selected from the group consisting of fluorine, chlorine, bromine and iodine. Particular embodiments of compounds of this class include compounds where a R₁, R₂, R₃, R₄, R₅ or R₆ that is neither H nor CH₃ is: —NR^{a—(CR}^{b}R^{c})ₙ—X wherein X is a halogen selected from the group consisting of chlorine, bromine and iodine, or is a water soluble group R^{a}, R^{b} and ^{RC} are, independently of each other, selected from the group consisting of hydrogen and optionally substituted hydrocarbyl, and n is an integer from 0 to 20.

Preferred examples of compounds of this class are:

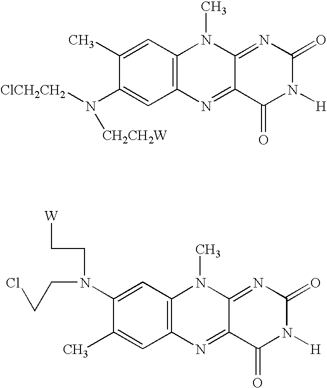

where W is a substituent imparting water solubility to the molecule, including, but not limited to, ascorbate, alcohol, polyalcohol; amine or polyamines, straight chain or cyclic saccharides, sulfates, phosphates, alkyl chains optionally substituted with —OH at any position, glycols, including polyethylene glycol and polyethers.

Another particular embodiment of compounds wherein a R₁, R₂, R₃, R₄, R₅ or R₆ that is neither H nor CH₃ contains a halogen or is a halogen includes compounds wherein a R₁, R₂, R₃, R₄, R₅ or R₆ that is neither H nor CH₃ is: X—(CH₂)ₙ—, wherein X is a halogen selected from the group consisting of chlorine, bromine and iodine, and n is an integer from 0 to 6. A preferred example of compounds of this class include:

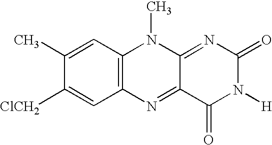

Other classes of compounds of this invention include those wherein R₁ is CH₂—(CH₂OH)₃—CH₂OH and those wherein R₁ is not CH₂—(CH₂OH)₃—CH₂OH. Also, those compounds wherein R₃ and R₆ are H are included in the invention.

A “carbonyl compound” is any compound containing a carbonyl group (—C═O). The term “amine” refers to a primary, secondary, or tertiary amine group. A “polyamine” is a group that contains more than one amine group. A “sulfate” group is a salt of sulfuric acid. Sulfate groups include the group (SO₄)²⁻. “Phosphates” contain the group PO₄³⁻. “Glycols” are groups that have two alcohol groups per molecule of the compound. “Glycols” are also known as dials. A glycol is described by the formula: CₙH₂ₙ(OH)₂, where n is an integer. An “aldehyde” is a group containing the formula —(C═O)—H. A “ketone” is a group with formula R—(C═O)—R, where R is not hydrogen. The R groups on ketone do not need to be the same. A “carboxylic acid” is a group which includes the formula: —COOH. An “ether” is a group containing —O—. A “salt” is a group where a hydrogen atom of an acid has been replaced with a metal atom or a positive radical, such as NH₄⁺. “Ascorbate” includes groups with formula:

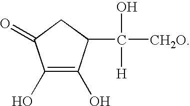

The term “hydrocarbyl” is used herein to refer generally to organic groups comprised of carbon chains to which hydrogen and optionally other elements are attached. CH₂ or CH groups and C atoms of the carbon chains of the hydrocarbyl may be replaced with one or more heteroatoms (i.e., non-carbon atoms). Suitable heteroatoms include but are not limited to O, S, P and N atoms. The term hydrocarbyl includes, but is not limited to alkyl, alkenyl, alkynyl, ether, polyether, thioether, straight chain or cyclic saccharides, ascorbate, aminoalkyl, hydroxylalkyl, thioalkyl, aryl and heterocyclic aryl groups, optionally substituted isoalloxazine molecules, amino acid, polyalcohol, glycol, groups which have a mixture of saturated and unsaturated bonds, carbocyclic rings and combinations of such groups. The term also includes straight-chain, branched-chain and cyclic structures or combinations thereof. Hydrocarbyl groups are optionally substituted. Hydrocarbyl substitution includes substitution at one or more carbons in the group by moieties containing heteroatoms. Suitable substituents for hydrocarbyl groups include but are not limited to halogens, including chlorine, fluorine, bromine and iodine, OH, SH, NH₂, C.H., CO₂H, ORₐ, SRₐ, NRₐR_{b}, CONRₐR_{b}, where Rₐ and R_{b} independently are alkyl, unsaturated alkyl or aryl groups.

The term “alkyl” takes its usual meaning in the art and is intended to include straight-chain, branched and cycloalkyl groups. The term includes, but is not limited to, methyl, ethyl, n-propyl, isopropyl, n-butyl, sec-butyl, isobutyl, tert-butyl, n-pentyl, neopentyl, 2-methylbutyl, 1-methylbutyl, 1-ethylpropyl, 1,1-dimethylpropyl, n-hexyl, 1-methylpentyl, 2-methylpentyl, 3-methylpentyl, 4-methylpentyl, 3,3-dimethylbutyl, 2,2-dimethylbutyl, 1,1-dimethylbutyl, 2-ethylbutyl, 1-ethylbutyl, 1,3-dimethylbutyl, n-heptyl, 5-methylhexyl, 4-methylhexyl, 3-methylhexyl, 2-methylhexyl, 1-methylhexyl, 3-ethylpentyl, 2-ethylpentyl, 1-ethylpentyl, 4,4-dimethylpentyl, 3,3-dimethylpentyl, 2,2-dimethylpentyl, 1,1-dimethylpentyl, n-octyl, 6-methylheptyl, 5-methylheptyl, 4-methylheptyl, 3-methylheptyl, 2-methylheptyl, 1-methylheptyl, 1-ethylhexyl, 1-propylpentyl, 3-ethylhexyl, 5,5-dimethylhexyl, 4,4-dimethylhexyl, 2,2-diethylbutyl, 3,3-diethylbutyl, and 1-methyl-1-propylbutyl. Alkyl groups are optionally substituted. Lower alkyl groups are C₁-C₆ alkyl and include among others methyl, ethyl, n-propyl, and isopropyl groups.

The term “cycloalkyl” refers to alkyl groups having a hydrocarbon ring, particularly to those having rings of 3 to 7 carbon atoms. Cycloalkyl groups include those with alkyl group substitution on the ring. Cycloalkyl groups can include straight-chain and branched-chain portions. Cycloalkyl groups include but are not limited to cyclopropyl, cyclobutyl, cyclopentyl, cyclohexyl, cycloheptyl, cyclooctyl, and cyclononyl. Cycloalkyl groups can optionally be substituted.

Aryl groups may be substituted with one, two or more simple substituents including, but not limited to, lower alkyl, e.g., methyl, ethyl, butyl; halo, e.g., chloro, bromo; nitro; sulfato; sulfonyloxy; carboxy; carbo-lower-alkoxy, e.g., carbomethoxy, carbethoxy; amino; mono- and di-lower-alkylamino, e.g., methylamino, ethylamino, dimethylamino, methylethylamino; amido; hydroxy; lower-alkoxy, e.g., methoxy, ethoxy; and lower-alkanoyloxy, e.g., acetoxy.

The term “unsaturated alkyl” group is used herein generally to include alkyl groups in which one or more carbon-carbon single bonds have been converted to carbon-carbon double or triple bonds. The term includes alkenyl and alkynyl groups in their most general sense. The term is intended to include groups having more than one double or triple bond, or combinations of double and triple bonds. Unsaturated alkyl groups include, without limitation, unsaturated straight-chain, branched or cycloalkyl groups. Unsaturated alkyl groups include without limitation: vinyl, allyl, propenyl, isopropenyl, butenyl, pentenyl, hexenyl, hexadienyl, heptenyl, cyclopropenyl, cyclobutenyl, cyclopentenyl, cyclopentadienyl, cyclohexenyl, cyclohexadienyl, 1-propenyl, 2-butenyl, 2-methyl-2-butenyl, ethynyl, propargyl, 3-methyl-1-pentynyl, and 2-heptynyl. Unsaturated alkyl groups can optionally be substituted.

Substitution of alkyl, cycloalkyl and unsaturated alkyl groups includes substitution at one or more carbons in the group by moieties containing heteroatoms. Suitable substituents for these groups include but are not limited to OH, SH, NH₂,CH, CO₂H, OR_{c}, SR_{c}, P, PO, NR_{c}R_{d}, CONR_{c}R_{d}, and halogens, particularly chlorines and bromines where R_{c} and R_{d}, independently, are alkyl, unsaturated alkyl or aryl groups. Preferred alkyl and unsaturated alkyl groups are the lower alkyl, alkenyl or alkynyl groups having from 1 to about 3 carbon atoms.

The term “aryl” is used herein generally to refer to aromatic groups which have at least one ring having a conjugated pi electron system and includes without limitation carbocyclic aryl, aralkyl, heterocyclic aryl, biaryl groups and heterocyclic biaryl, all of which can be optionally substituted. Preferred aryl groups have one or two aromatic rings.

“Carbocyclic aryl” refers to aryl groups in which the aromatic ring atoms are all carbons and includes without limitation phenyl, biphenyl and napthalene groups.

“Aralkyl” refers to an alkyl group substituted with an aryl group. Suitable aralkyl groups include among others benzyl, phenethyl and picolyl, and may be optionally substituted. Aralkyl groups include those with heterocyclic and carbocyclic aromatic moieties.

“Heterocyclic aryl groups” refers to groups having at least one heterocyclic aromatic ring with from 1 to 3 heteroatoms in the ring, the remainder being carbon atoms. Suitable heteroatoms include without limitation oxygen, sulfur, and nitrogen. Heterocyclic aryl groups include among others furanyl, thienyl, pyridyl, pyrrolyl, N-alkyl pyrrolo, pyrimidyl, pyrazinyl, imidazolyl, benzofuranyl, quinolinyl, and indolyl, all optionally substituted.

“Heterocyclic biaryl” refers to heterocyclic aryls in which a phenyl group is substituted by a heterocyclic aryl group ortho, meta or para to the point of attachment of the phenyl ring to the decalin or cyclohexane. Heterocyclic biaryl includes among others groups which have a phenyl group substituted with a heterocyclic aromatic ring. The aromatic rings in the heterocyclic biaryl group can be optionally substituted.

“Biaryl” refers to carbocyclic aryl groups in which a phenyl group is substituted by a carbocyclic aryl group ortho, meta or para to the point of attachment of the phenyl ring to the decalin or cyclohexane. Biaryl groups include among others a first phenyl group substituted with a second phenyl ring ortho, meta or para to the point of attachment of the first phenyl ring to the decalin or cyclohexane structure. Para substitution is preferred. The aromatic rings in the biaryl group can be optionally substituted.

Aryl group substitution includes substitutions by non-aryl groups (excluding H) at one or more carbons or where possible at one or more heteroatoms in aromatic rings in the aryl group. Unsubstituted aryl, in contrast, refers to aryl groups in which the aromatic ring carbons are all substituted with H, e.g. unsubstituted phenyl (—C₆H₅), or naphthyl (—C₁₀H₇). Suitable substituents for aryl groups include among others, alkyl groups, unsaturated alkyl groups, halogens, OH, SH, NH₂, C.H., CO₂H, ORₑ, SRₑ, NRₑR_{f}, CONRₑR_{f}, where Rₑ and R_{f} independently are alkyl, unsaturated alkyl or aryl groups. Preferred substituents are OH, SH, ORₑ, and SRₑ where Rₑ is a lower alkyl, i.e., an alkyl group having from 1 to about 3 carbon atoms. Other preferred substituents are halogens, more preferably chlorine or bromine, and lower alkyl and unsaturated lower alkyl groups having from 1 to about 3 carbon atoms. Substituents include bridging groups between aromatic rings in the aryl group, such as —CO₂—, —CO—, —O—, —S—, —P—, —NH—, —CH═CH— and —(CH₂)ₗ— where l is an integer from 1 to about 5, and particularly —CH₂—. Examples of aryl groups having bridging substituents include phenylbenzoate. Substituents also include moieties, such as —(CH₂)ₗ—, —O—(CH₂)ₗ— or —OCO—(CH₂)ₗ—, where l is an integer from about 2 to 7, as appropriate for the moiety, which bridge two ring atoms in a single aromatic ring as, for example, in a 1, 2, 3, 4-tetrahydronaphthalene group. Alkyl and unsaturated alkyl substituents of aryl groups can in turn optionally be substituted as described supra for substituted alkyl and unsaturated alkyl groups.

The terms “alkoxy group” and “thioalkoxy group” (also known as mercaptide groups, the sulfur analog of alkoxy groups) take their generally accepted meaning. Alkoxy groups include but are not limited to methoxy, ethoxy, n-propoxy, isopropoxy, n-butoxy, sec-butoxy, isobutoxy, tert-butoxy, n-pentyloxy, neopentyloxy, 2-methylbutoxy, 1-methylbutoxy, 1-ethyl propoxy, 1,1-dimethylpropoxy, n-hexyloxy, 1-methylpentyloxy, 2-methylpentyloxy, 3-methylpentyloxy, 4-methylpentyloxy, 3,3-dimethylbutoxy, 2,2-dimethoxybutoxy, 1-1-dimethylbutoxy, 2-ethylbutoxy, 1-ethylbutoxy, 1,3-dimethylbutoxy, n-pentyloxy, 5-methylhexyloxy, 4-methylhexyloxy, 3-methylhexyloxy, 2-methylhexyloxy, 1-methylhexyloxy, 3-ethylpentyloxy, 2-ethylpentyloxy, 1-ethylpentyloxy, 4,4-dimethylpentyloxy, 3,3-dimethylpentyloxy, 2,2-dimethylpentyloxy, 1,1-dimethylpentyloxy, n-octyloxy, 6-methylheptyloxy, 5-methylheptyloxy, 4-methylheptyloxy, 3-methylheptyloxy, 2-methylheptyloxy, 1-methylheptyloxy, 1-ethylhexyloxy, 1-propylpentyloxy, 3-ethylhexyloxy, 5,5-dimethylhexyloxy, 4,4-dimethylhexyloxy, 2,2-diethylbutoxy, 3,3-diethylbutoxy, 1-methyl-1-propylbutoxy, ethoxymethyl, n-propoxymethyl, isopropoxymethyl, sec-butoxymethyl, isobutoxymethyl, (1-ethyl propoxy)methyl, (2-ethylbutoxy)methyl, (1-ethylbutoxy)methyl, (2-ethylpentyloxy)methyl, (3-ethylpentyloxy)methyl, 2-methoxyethyl, 1-methoxyethyl, 2-ethoxyethyl, 3-methoxypropyl, 2-methoxypropyl, 1-methoxypropyl, 2-ethoxypropyl, 3-(n-propoxy)propyl, 4-methoxybutyl, 2-methoxybutyl, 4-ethoxybutyl, 2-ethoxybutyl, 5-ethoxypentyl, and 6-ethoxyhexyl. Thioalkoxy groups include but are not limited to the sulfur analogs of the alkoxy groups specifically listed supra.

“Optional” or “optionally” means that the subsequently described event or circumstance may or may not occur, and that the description includes instances where said event or circumstance occurs and instances in which it does not. For example, “optionally substituted phenyl” means that the phenyl radical may or may not be substituted and that the description includes both unsubstituted phenyl radicals and phenyl radicals wherein there is substitution.

“Amino acids” as used herein include naturally occurring and commercially available amino acids and optical isomers thereof. Typical natural and commercially available amino acids are glycine, alanine, serine, homoserine, threonine, valine, norvaline, leucine, isoleucine, norleucine, aspartic acid, glutamic acid, lysine, ornithine, histidine, arginine, cysteine, homocysteine, methionine, phenylalanine, homophenylalanine, phenylglycine, o-, m-, and p-tyrosine, tryptophan, glutamine, asparagine, proline and hydroxyproline. “Amino acid” as used herein includes amino acid residues and amino acid side chains. An “amino acid residue” is an amino acid radical —NHCH(R)C(O)—, wherein R is an amino acid side chain, except for the amino acid residues of proline and hydroxyproline which are —N(CH₂—CH₂—CH₂)CHC(O)— and —N(CH—CHOHCH₂)CHC(O)—, respectively. An amino acid side chain is a radical found on the a-carbon of an a-amino acid as defined herein, where the radical is either hydrogen (side chain of glycine), methyl (side chain of alanine), or is a radical bonded to the a-carbon by a methylene (—CH₂-29), or phenyl group.

A protected glucose derivative takes its usual meaning in the art and includes a glucose molecule wherein some of the hydroxyl groups are substituted with acetate groups.

“Straight chain or cyclic saccharides” include mono-, di- and poly-, straight chain and cyclic saccharides that are optionally substituted with an amino group which is optionally acetylated. Straight chain saccharides that are useful in this invention include but are not limited to those molecules with a chain of 5 or 6 carbon atoms with one or more —OH groups attached, and either an aldehyde or ketone group. Cyclic saccharides are saccharides that are in a ring form. Disaccharides are compounds wherein two monosaccharide groups are linked. Polysaccharides are compounds wherein more than two monosaccharide groups are linked. Specific examples of saccharides useful in this invention include glucose, ribose and glucosamine, among others.

“Isoalloxazine”, “isoalloxazine derivative” or “core structure of isoalloxazine” include compounds that comprise the structure:

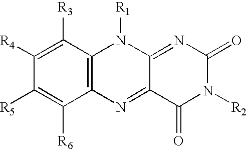

where R₁-R₆ are substituted with various substituents, as described elsewhere.

As used herein, the term “neutralization of a microorganism” or “neutralizing” means totally or partially preventing the microorganism from replicating, either by killing the microorganism or otherwise interfering with its ability to reproduce. A “neutralizer” is a compound that is capable of neutralizing a microorganism. The neutralizers useful in this invention include molecules with the core structure of isoalloxazine, as defined above. To “activate the microorganism neutralizer” is to expose the microorganism neutralizer to a triggering event that causes it to become active toward neutralizing microorganisms.

“Triggering event” refers to the stimulus that activates the microorganism neutralizer. Preferred triggering events include exposure of the neutralizer to an neutralization effective wavelength of light, or a pH sufficient to activate the neutralizer to neutralize microorganisms.

“Water soluble group” includes a group that, when included as a substituent on the neutralizer, imparts substantial solubility in water to the compound. Typically, the compound is soluble in water at a concentration of about 10-150 μM. Water soluble groups as referred to in this invention include, but are not limited to alcohols; polyalcohols; straight chain or cyclic saccharides; amines and polyamines; sulfate groups; phosphate groups; ascorbate groups; alkyl chains optionally substituted with —OH at any position; glycols, including polyethylene glycols, and polyethers.

The term “biologically active” means capable of effecting a change in a living organism or component thereof. “Biologically active” with respect to “biologically active protein” as referred to herein does not refer to proteins which are part of the microorganisms being neutralized. Similarly, “non-toxic” with respect to the neutralizers means low or no toxicity to humans and other mammals, and does not mean non-toxic to the microorganisms being neutralized. “Substantial destruction” of biological activity means at least as much destruction as is caused by porphyrin and porphyrin derivatives, metabolites and precursors which are known to have a damaging effect on biologically active proteins and cells of humans and mammals. Similarly, “substantially non-toxic” means less toxic than porphyrin, porphyrin derivatives, metabolites and precursors that are known for blood sterilization.

“Decomposition” of the neutralizer upon exposure to light refers to the chemical transformation of the neutralizer into new compounds. An example of decomposition of the neutralizer is the production of lumichrome upon exposure of riboflavin to visible light.

A “photosensitizer” is defined as any compound which absorbs radiation of one or more defined wavelengths and subsequently utilizes the absorbed energy to carry out a chemical process. Photosensitizers of this invention may include compounds which preferentially adsorb to nucleic acids, thus focusing their photodynamic effect upon microorganisms and viruses with little or no effect upon accompanying cells or proteins. Other photosensitizers of this invention are also useful, such as those using singlet oxygen-dependent mechanisms.

An “alkylating agent” is a compound that reacts with amino acid residues and nucleic bases and inhibits replication of microorganisms.

The terms “photoactivator” and “photosensitizer” are used synonymously herein.

“Substantial destruction” of biological activity means at least as much destruction as is caused by porphyrin and porphyrin derivatives, metabolites and precursors of which are known to have a damaging effect on biologically active proteins and cells of humans and mammals. Similarly, “substantially non-toxic” means less toxic than porphyrin, porphyrin derivatives, metabolites and precursors that are known for blood sterilization.

As is known to the art, platelets collected from patients typically include a plasma component. The ratios of platelets:storage solution referred to herein refers to the ratio of platelets and their associated plasma to storage solution.

The amount of photosensitizer (also referred to herein as “neutralizer”) to be mixed with the fluid will be an amount sufficient to adequately inactivate microorganisms therein, but less than a toxic (to humans or other mammals) or insoluble amount. Excess photosensitizer may be used as long as the concentration is not so high that the photosensitizer prevents light from passing to the desired depth at the required intensity. Optimal concentrations for desired photosensitizers may be readily determined by those skilled in the art without undue experimentation. Preferably the photosensitizer is used in a concentration of at least about 1 micromolar. The optimum concentration of photosensitizer will vary depending on the blood component being treated and the level to which plasma is removed. If red blood cells are being treated, a higher concentration of photosensitizer is desired than if platelets are being treated. If red blood cells are being treated with riboflavin, a useful concentration of riboflavin is about 1-200 micromolar, and a preferred concentration of riboflavin is about 50 to 150 micromolar when the plasma content is about 0 to 5% of the total volume of the solution. If platelets are being treated, a useful concentration of riboflavin is about 1-100 micromolar, and a preferred concentration of riboflavin is about 10 to 50 micromolar when the storage solution content is about 10-90% of the total volume of the solution.

The dissolved oxygen content of the fluid to be decontaminated should be greater than the amount which would normally be present in the fluid as a result of contact with the atmosphere (in the absence of mixing or increasing the oxygen content of the atmosphere in immediate contact with the fluid). Preferably the oxygen concentration in the fluid should be great enough to measurably increase the inactivation of microorganisms in the fluid, but not so great as to significantly damage cellular blood components, or desired biologically-active components such as proteins. It is typically useful to increase the dissolved oxygen content of the fluid to between about 1 and about 5 times the dissolved oxygen content that would be present in the fluid under an air atmosphere at ambient temperature and pressure without mixing. In one embodiment of this invention the dissolved oxygen content of the fluid is increased to about five times the dissolved oxygen content that would be present in said fluid under an air atmosphere at ambient temperature and pressure without mixing. The oxygen content of the fluid may be increased by any means known to the art, and can be increased by mixing the fluid with air, such as by vigorous agitation at a mixing speed of between about 70 and about 150 cpm using a linear shaker for between about 1 and about 5 minutes, or by adding oxygen directly to the atmosphere in contact with the fluid, e.g. to a blood component bag containing the fluid. Preferably about two or three times the amount of oxygen needed to saturate the fluid is provided, and the solution is allowed to reach equilibrium with the atmosphere. Pure medical grade oxygen gas may be used. The volume of gas to be added and method of oxygen addition may be readily determined by one skilled in the art. To further increase the dissolved oxygen content of the fluid when oxygen is being added, mixing can be used as above. Hyperbaric pressure on the fluid may also be used to increase dissolved oxygen pressure in the presence of air or other oxygen-containing gases, or pure oxygen or such gases can be bubbled through the fluid. Oxygen may be continuously added to the fluid during photoradiation, or may be replenished from time to time to the extent this is required to avoid reactions of the photosensitizer which would prevent it from being recycled.

When excess oxygen and excess photoactivator are used, the reaction ratio can be controlled by use of more or less light.

To prevent damage to cellular blood components or other desired biologically-active components of the fluid, a lipophilic antioxidant may also be added to said fluid in an amount effective to substantially prevent damage to desired biological components of said fluid (not including pathogenic microorganisms). The lipophilic moieties of the antioxidant target it to platelet cell walls to aid in protection of cells. Addition of such lipophilic antioxidants to the system does not adversely affect pathogen inactivation. Suitable lipophilic antioxidants include cysteine derivatives such as N-acetyl-L-cysteine, N-acetyl-D-cysteine (NAC), glutathione (GSH), L-cysteine, as well as butylated hydroxyanisole (BHA), nordihydroguaiaretic acid (NDGA), dithiocarbamates, lipoic acid, and Vitamin E, vitamin E derivatives such as vitamin E succinate, ascorbate, and preferably Vitamin E. The lipophilic antioxidant should be present in the fluid in an amount sufficient to be available for all cells to be protected, but not so much as to become insoluble or interfere with viability of cellular biological components being decontaminated or otherwise interfere with the process of this invention. Preferably, the lipophilic antioxidant is present in the fluid in an amount between about 0.25 mg/ml and about 2 mg/ml, more preferably between about 0.5 mg/ml and about 1 mg/ml.

A metal chelator may also be present in amounts sufficient to provide a cell-protective effect while not interfering with the claimed process. Such chelators include DTC (dithiocarb sodium, Imuthiol).

The fluid is then irradiated with light, preferably visible light, at a photoradiation energy sufficient to activate the photosensitizer and provide measurable pathogen inactivation, but not so much as to substantially convert photosensitizer present to non-photoactive compounds, e.g. to not convert riboflavin to lumichrome, before pathogen reduction is achieved. When sufficient oxygen is present in the system, excess light energy will not be harmful. Preferably the energy of photoradiation is between about 5 and about 15 J/cm², more preferably between about 10 and about 12 J/cm². The photoradiation is continued for a period of time sufficient to substantially inactivate microorganisms in said fluid, preferably for about two to about 15 minutes, more preferably for about five to about seven minutes.

Microorganisms inactivated by the present method may be selected from the group consisting of extracellular and intracellular viruses, bacteria, bacteriophages, fungi, blood-transmitted parasites, and protozoa, and mixtures of any two or more of the foregoing. For example, viruses inactivated by the present method may be selected from the group consisting of acquired immunodeficiency (HIV) virus, hepatitis A, B and C viruses, sindbis virus, cytomegalovirus, vesicular stomatitis virus, herpes simplex viruses, e.g. types I and II, human T-lymphotropic retroviruses, HTLV-III, lymphadenopathy virus LAV/IDAV, parvovirus, transfusion-transmitted (TT) virus, and Epstein-Barr virus, bovine viral diarrhea virus, pseudorabies, and mixtures of any two or more of the foregoing. Bacteriophages inactivated by the present process may be selected from the group consisting of ΦX174, Φ6, λ, R₁₇, T₄, and T₂, and mixtures of any two or more of the foregoing. Bacteria may be selected from the group consisting of P. aeruginosa, S. aureus, S. epidermidis, E. coli, K. pneumoniae, E. faecalis, B. subtilis, S. pneumoniae, S. pyrogenes, S. viridans, B. cereus, E aerogenes, propionabacter, C. perfringes, E. cloacae, P. mirabilis, S. cholerasuis, S. liquifaciens, S. mitis, Y. entercolitica, P. fluorescens, S. enteritidis, C. freundii, and S. marcescens, and mixtures of any two or more of the foregoing.

This invention also provides methods for treating platelets to inactivate microorganisms which may be present therein, comprising:

(a) mixing 7,8-dimethyl-10-ribityl isoalloxazine with a fluid comprising said platelets in storage solution at a ratio of between about 20:80 and about 90:10 platelets:storage solution, whereby the 7,8-dimethyl-10-ribityl isoalloxazine concentration of said fluid is between about 1 and about 200 micromolar, preferably about 50 micromolar.

(b) increasing the dissolved oxygen content of said fluid to about five times that oxygen content of said fluid under an air atmosphere, by mixing air into said fluid, or exposing said fluid to an atmosphere of substantially pure oxygen;

(c) exposing said fluid to photoradiation at an energy between about 5 and about 15 J/cm² to activate the photosensitizer, for at least about 3 to about 7 minutes, to substantially inactivate said microorganisms.

The method may also comprise adding vitamin E to the fluid. Again, preferably the fluid comprises a blood product.

This invention also provides blood products decontaminated by the foregoing methods.

After treatment according to the methods of this invention, blood or blood product or other fluids may be delivered to a patient, concentrated, or infused directly.

This invention further comprises biological compositions comprising:

(a) a fluid;

(b) an inactivation-effective, substantially non-toxic amount of an endogenous photosensitizer or endogenously-based derivative photosensitizer;

(c) dissolved oxygen in said fluid in an amount greater than would be present under an air atmosphere at ambient conditions without mixing.

The compositions of this invention comprise fluids, photosensitizers, and concentrations of components as described above, and may also comprise additives such as lipophilic antioxidants, as described above with respect to the methods of this invention. Such compositions contained within a blood component bag or other suitable container known to the art for processing or storage are also provided by this invention. The blood component bag or other container may also comprise an internal gas comprising a larger-than-atmospheric amount of oxygen. The blood component bag may comprise substantially pure oxygen. “Substantially pure” oxygen is oxygen as transferred from an art-known commercial oxygen tank using commercially available standard connections.

Blood component bags are known to the art and generally have a volume of between about 100 and about 1000 ml, although they may have a volume up to about 3000 ml. Preferably the volume of fluid in the blood component bags used in this invention is between about 100 and about 600 ml, more preferably between about 250 and about 350 ml.

This invention also comprises a decontamination system for a fluid comprising:

(a) a leak-proof transparent or translucent container for the fluid;

(b) a photosensitizer source for providing photosensitizer to said container, said photosensitizer source being connectible to an inlet of said container;

(c) an oxygen source connectible to an inlet of said container for providing oxygen to said container; and

(d) a photoirradiator for irradiating said container.

Such, leak-proof containers are known to the art and include blood component bags used for collection and storage. The container may be gas-tight or semi-permeable to gas. Semi-permeable containers are preferred for use with platelets, since long-term platelet storage requires a breathable container. This is not true for red blood cells, for which gas-tight containers can be used. When a semi-permeable container is used, photoradiation should take place soon enough after photoirradiation that the atmosphere in the container has not equilibrated with the outside atmosphere. The container should be transparent to light or sufficiently translucent to allow passage therethrough of sufficient photoradiation to activate the photosensitizer to provide measurable pathogen inactivation. In the system provided by this invention, the container for the fluid may comprise a blood product selected from the group consisting of whole blood, platelets, plasma, and red blood cells. Preferably, the blood product is platelets or red blood cells, and more preferably consists essentially of platelets in storage solution at a ratio between about 20:80 and about 90:10 platelets:storage solution.

The photosensitizer source may be a container for the photosensitizer in powder or fluid form, and preferably also comprises means (e.g. automated means which may be computer-controlled) for adding photosensitizer to the container for the fluid, such as tubes connecting the photosensitizer source to the container for the fluid, preferably including means for metering the amount of photosensitizer added. Alternatively, photosensitizer may be added to the container for the fluid by hand, e.g. using syringes, droppers, and the like. Any means for adding the photosensitizer to the fluid to be decontaminated and for placing the fluid in the photopermeable container known to the art may be used, such means typically including flow conduits, ports, reservoirs, sterile docking, valves, and the like. The system may include means such as pumps or adjustable valves for controlling the flow of the photosensitizer into the fluid to be decontaminated so that its concentration may be controlled at effective levels as described herein. In one embodiment, photosensitizer is mixed with the anticoagulant feed to a blood apheresis system. For endogenous photosensitizers and derivatives having sugar moieties, the pH of the solution is preferably kept low enough, i.e. between about 3 and about 5, as is known to the art, to prevent detachment of the sugar moiety. Preferably the photosensitizer is added to the fluid to be decontaminated in a pre-mixed aqueous solution, e.g., in saline or buffer solution.

The system of this invention may include a photosensitizer source which contains a photosensitizer as described above.

The photosensitizer and any optional desired additives may be placed in a container as dried medium, including powder or pill form, or as a solution. Desired additives include nutrients or other materials such as acetate, glucose, dextrose, citrate, pyruvate, potassium, or magnesium, which allow the components to retain biological activity or improve the storage lifetime. It may be desirable for platelets to be provided nutrients when the storage solution concentration is less than about 20% of the total volume of the sample in order for the platelets to remain active. Desired additives and the photosensitizer may be sterilized as powders. In one embodiment, the powders desired are placed in the container prior to introduction of fluids to be decontaminated.

If the photosensitizer and any desired additives are placed in the container as one or more solutions, the volume and composition of the solution(s) may produce the desired percentage of storage solution in the sample without further additions of solution, or the percentage of storage solution may be adjusted before, during or after placing said fluid in said container. Adjustment of the percentage of storage solution after placing the fluid in the container may occur by the introduction of a suitable solution after the fluid is in the container. Adjustment of the percentage of storage solution may occur during introduction of the fluid in a container by the introduction of a suitable solution as the fluid is being placed in the container. To determine the amount of solution to be added, the containers may be weighed, or evaluated by eye or other measuring instrument known to the art.

The oxygen source may be any oxygen source known to the art, e.g. an oxygen tank, and preferably includes means (e.g. automated means which may be computer-controlled) for adding oxygen to the container for the fluid, such as tubes equipped with leak-proof valves connecting the oxygen source to the container for the fluid. Preferably the means for adding oxygen include means for metering the amount of oxygen added. Preferably the decontamination system also comprises a sterile barrier between the oxygen source and the container, as is known to the art, such as a sterile barrier filter.

The system of this invention may include a container for the fluid which contains a fluid to be decontaminated. The container may also contain a fluid having a greater-than-normal oxygen concentration. The container may also contain a fluid including a photosensitizer. The container may also contain an atmosphere containing a higher-than-normal oxygen concentration.

The container may be placed in a rack for irradiation or upon a flat surface, or shaker table.

The inlets to the container for the fluid through which photosensitizer and oxygen are added may be the same or may be different.

The photoirradiator for irradiating the container may be any device or collection of components known to the art for shining light on the fluid within the container. Some examples of light sources that may be used include the following: a Philips “Special Blue” F20T12/BB 20 watt light which emits wavelengths from about 400 to 500 nm; Ultraviolet Resources International's URI FR20T12 super actinic/VHO-1 CE U123 lamp which emits wavelengths from about 400 nm to about 450 nm; Custom Sea Life “Power Compact” 7100K Blue 28 watt Twin Tube which emits wavelengths from about 400 nm to about 520 nm. The Sylvania “Blue” F20T 12B 20 watt bulb which has a broad emission from about 400 nm to about 640 nm. Other representative light sources that may be used include Philips PL-L-36W with peak output at a wavelength of about 365 nm. Super Actinic lamps generally have a spectral range from about 400 to about 440 nm, with a peak at 420 nm and may be used herein. Bilirubin lights used to treat infants suffering from jaundice may also be used. For example, a light from Philips Lighting having a peak output at 447 nm and a range of about 420-460 nm may be used.

Lights that emit in the desired spectral range come from various sources. Lamps with peak emissions around 420 to 450 nm may be purchased from LCD Lighting, Orange, Conn.; Bulbtronic, Farmingdale, N.Y.; National Biological Corp., Twinsburg, Ohio; The Fluorescent Co., Saugus, Calif.; Tek-West, Los Angeles, Calif.; or Southern Nebr. UV, Bransford, Conn., for example. LED (light emitting diodes) may also be used. These LEDs may use a variety of materials to produce the desired spectral output, including silicon carbide (bandwidth around 100 nm; peak spectral output near 466 nm) or gallium nitride (bandwidth around 30-35 nm; peak spectral output near 470 nm). Also, lights made from a combination of different materials can generate different wavelengths of light. For example, gallium nitride on a silicon carbide substrate can generate 430 nm. These LEDs are manufactured or distributed by Panasonic, Chicago Miniature, Nichia Co. (Tokushima, JP) Toyoda Gosei, Hewlett Packard, and LEDTronics, for example. LED devices are also supplied by Cree, Inc. (Durham, N.C.), Kingbright Corp. (City of Industry, Calif.) and Limileds Lighting, LLC (San Jose, Calif.). LED lights typically do not require any outside cooling.

Pulsed lights may be used, and irradiation performed as set forth in U.S. patent application Ser. No. 09/962,029 filed Sep. 25, 2001, incorporated by reference herein to the extent not inconsistent herewith.

Visible and/or ultraviolet light sources may be used.

The lights may be used in different ways, depending the particular apparatus. For example, arrays of diodes may surround the fluid in any desired configuration. In a flat bed apparatus, light arrays may surround the fluid from top or bottom, or both.

Filters, such as colored glass filters, ultraviolet light filters, or mylar filters, may be used to isolate a desired band of the spectrum or adjust the amount of irradiation. Single wavelength or narrow band light sources may also be used.

One embodiment of an apparatus useful in the methods of the invention includes banks of interchangeable lights that produce the desired wavelength of light for the particular fluid being treated. A super actinic lamp or a blue LED may be used to produce 419 nm light that is useful in inactivating microorganisms in platelets. Coral or aquarium lights may be used to produce wavelengths between 400 and 500 nm that is useful in inactivating microorganisms in red blood cells. The lamps may be provided with separate power supplies to control the level of light output.

Active (cooling through some applied means) or passive (air cooling) cooling may be used if necessary to cool either the lamps or the fluid, e.g. the blood component. Fans may provide cooling. One set of fans may be used to cool both the lamps and fluid, or different fans may be used to provide different levels of cooling to both the lamps and the fluid. A photopermeable liquid or gas may surround the sample and/or lights to provide active cooling. This liquid or gas may be optionally temperature controlled.

The decontamination system of this invention may comprise an agitator for agitating the container as described above, used instead of the oxygen source, or in addition to the oxygen source to increase the oxygen concentration of the fluid. Preferably the decontamination system also comprises a sterile barrier between the oxygen source and the container. The system may also comprise a scale for weighing the container. A bar-coded label for the container and a scanner for reading the bar-coded label, as well as a computer processor for receiving, correlating and storing data identifying the container, the weight of said container, and the fact that said container has been irradiated may also be included as components of the system of this invention. One or more apparatuses which can be set at room temperature and agitate, e.g. shake or rotate, the container containing the product to be irradiated, such as the Helmer platelet incubator/agitator (Helmer Company, Noblesville, Ind.) for placing containers before and/or after irradiation may also be included in the system.

Decontamination systems as described above may be designed as stand-alone units or may be incorporated into existing apparatuses known to the art for separating or treating blood being withdrawn from or administered to a patient. Such blood-handling apparatuses include, for example, the GAMBRO Spectra™ or TRIMA® apheresis systems, available from GAMBRO Inc., Lakewood, Colo., or the apparatuses described in U.S. Pat. No. 5,653,887, U.S.

Ser. No. 08/924,519 filed Sep. 5, 1997 (PCT Publication No. WO 99/11305) and PCT Publication No. WO 01/66172 of GAMBRO, Inc., as well as the apheresis systems of other manufacturers. The decontamination system may be inserted just downstream of the point where blood is withdrawn from a patient or donor, just prior to insertion of blood product into a patient, or at any point before or after separation of blood constituents. The storage solution may be adjusted at any point before fluid is exposed to irradiation. In other embodiments, decontamination systems of this invention may be used to process previously collected and stored blood products.

When red blood cells are present in the fluid being treated, as will be appreciated by those skilled in the art, to compensate for absorption of light by the cells, the fluid may be thinned, exposed to higher energies of radiation for longer periods, agitated for longer periods or presented to photoradiation in shallower containers or conduits than necessary for use with other blood components.

Methods of making such decontamination systems are also provided herein, comprising providing and assembling the above components, i.e.:

(a) providing a set of components comprising:

(i) a leak-proof transparent or translucent container for the fluid;

(ii) a photosensitizer source for providing photosensitizer to said container, said photosensitizer source being connectible to an inlet of said container;

(iii) an oxygen source connectible to an inlet of said container for providing oxygen to said container;

(iv) a photoirradiator for irradiating said container; and

(b) assembling said components in operational proximity to each other.

The method of making decontamination systems of this invention may also comprise connecting the photosensitizer source to the appropriate inlet of the container, and connecting the oxygen source to the appropriate inlet of the container.

This invention also provides methods of decontaminating a fluid comprising:

(a) providing a set of components comprising:

(i) a leak-proof transparent or translucent container for the fluid;

(ii) a photosensitizer source for providing photosensitizer to said container, said photosensitizer source being connectible to an inlet of said container;

(iii) an oxygen source connectible to an inlet of said container for providing oxygen to said container;

(iv) a photoirradiator for irradiating said container;

(b) assembling said components in operational proximity to each other;

(c) connecting said photosensitizer source to an inlet of said container;

(d) transferring photosensitizer from said photosensitizer source to said container;

(e) connecting said oxygen source to an inlet of said container;

(f) replacing the atmosphere in said container with oxygen from said oxygen source;

(g) positioning said container with respect to said irradiator such that radiation from said irradiator reaches fluid within said container;

(h) activating said irradiator to irradiate said fluid, thereby decontaminating said fluid.

The term “operational proximity” means that the components are arranged such that moving the fluid through the decontamination system may be done efficiently by automated means and/or by hand.

The method for decontaminating a fluid may also comprise providing an agitator to agitate said fluid within said container and activating said agitator to agitate said fluid. Agitation may be done prior to or simultaneous with irradiation of the container.

This invention further provides a method of increasing the storage life of photochemically decontaminated platelets comprising:

(a) placing said platelets in a container larger than the volume of a solution containing said platelets; and

(b) dissolving an amount of oxygen in said solution greater than that would be dissolved in said solution under an air atmosphere at ambient conditions without agitation;

(c) adding a photoactivator to said solution and irradiating said solution to activate said photoactivator;

(d) removing oxygen from the atmosphere in said container; and

(e) storing said platelets.

The platelets are preferably stored within said container.

Preferably the volume of the container is at least about twice as large as the volume of the solution to provide an oxygen-containing atmosphere above the solution. The volume of solution in the container should be large enough so as to cover the surface area to be exposed to the lights, but not so high as to create a light path so long it would diminish activation efficiency or prevent the addition of gas. Preferably the solution volume in the container is between about 250 ml and about 350 ml. The volume of atmosphere above the solution in the container should be large enough so as to cover the fluid surface, but not so high as to dramatically distort the container. Preferably the volume of atmosphere in the container is about 10% to about 50% the volume of the solution. The storage life of platelets may be increased by this method by about 20% to about 40%, e.g. from about five to about seven days.

The methods of this invention are performed at temperatures which would not result in damage to the cells. Preferably the temperature is not above about 45° C. and not below about 4° C. Most preferably, the temperature is between about body temperature, and about 28° C.

After performing the method, excess gaseous oxygen is preferably removed from the fluid before storing to prevent damage to platelets or other sensitive components.

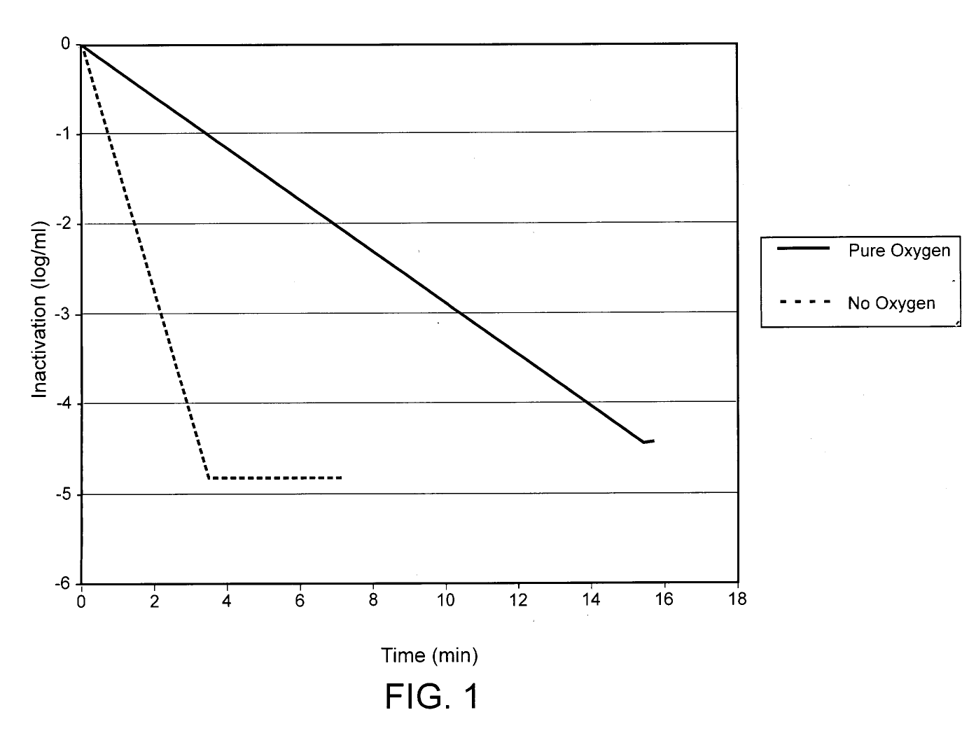

FIG. 1 is a graph showing BVDV inactivation in the presence of 50 micromolar riboflavin in 27% platelets in plasma with and without oxygen. In the presence of oxygen, inactivation is more than four times as fast, and significantly lower BVDV levels are achieved.

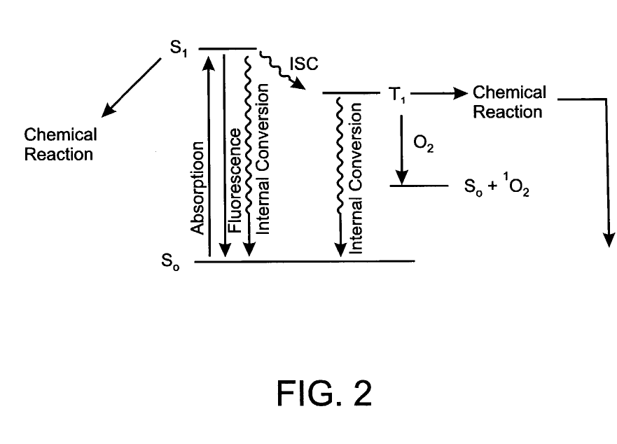

FIG. 2 is a Jablonski diagram showing chemical reactions of 7,8-dimethyl-10-ribityl isoalloxazine (riboflavin and related photoactivator compounds) catalyzed by photoradiation, oxygen and other components. As is known to the art, internal conversion is the radiationless transition between energy states of the same spin state. Intersystem crossing (ISC) is radiationless transition between different spin states. When the molecule relaxes from the singlet state to the ground state, it is called fluorescence; when it relaxes from the triplet state (S₁) to the ground (unexcited) state (S₀), this is called phosphorescence. The left arrow indicates that upon absorption of light energy (first vertical, upward-pointing, arrow) the molecule can go from its ground state to its excited state and become involved in chemical reactions including losing its ribityl moiety to become lumichrome (7,8-dimethylalloxazine). Lumichrome is not photoactive under visible light. Alternatively, as shown by the second vertical, downward pointing, arrow, the excited molecule may release its absorbed energy and fluoresce and return to the ground state. The wavy arrows indicate that energy is released. The wavy line labeled ISC indicates intersystem crossing wherein the molecule transfers to the triplet state (T₁) by changing the spin of an electron (spin conversion). If no oxygen is present, the molecule in its triplet state can phosphoresce (second wavy, downward pointing, arrow) and return to its ground state. Or, as indicated by the right arrow, the molecule in its triplet state can react with other molecules in close proximity including guanine and other proteins such as ascorbate or glutathione and return to its ground state. If oxygen is present, the molecule in its triplet state can react with oxygen and return to its ground state producing ¹O₂ (singlet oxygen), this being desirable for pathogen kill because singlet oxygen can effectively cause DNA strand breaks, contributing to pathogen kill.

Pathogen kill using riboflavin and related photosensitizer compounds occurs upon photoactivation via singlet oxygen damage, or via binding of the photosensitizer to nucleic acids of the pathogen, thereby disrupting the ability of the pathogen to function and reproduce, or both. Photosensitizer may not be recycled and reused in the system when irreversible reactions occur (such as the conversion of riboflavin to lumichrome which does not respond to visible light). If oxygen is present in the system, however, riboflavin may be sent down the singlet oxygen pathway, whereby singlet oxygen is produced and the photosensitizer molecule is recycled and returned to its ground state where it is again available for irradiation to produce the triplet state and can again react with oxygen to form more singlet oxygen. Alternatively, it can bind to proteins in the system. The formation of these riboflavin-protein adducts also removes riboflavin from the system and reduces the efficiency of the pathogen inactivation progress.

When oxygen is depleted in the system, irreversible reactions are favored: (1) reactions converting the photosensitizer to compounds which are not photoactive under visible light; and (2) binding reactions to proteins such that the photosensitizer is not free to effect further pathogen kill, both of which reactions remove photosensitizer as an active component of the system. More effective pathogen kill is therefore achieved when oxygen is added to favor reversible reactions in which the photosensitizer is recycled. Irradiating the fluid causes riboflavin to consume oxygen. When oxygen is not present in sufficient quantity, the irradiation process will consume the photoactivator. Addition of oxygen is therefore required to maintain production of singlet oxygen. Reducing light intensity (for low oxygen environments) helps to prevent the conversion of riboflavin to a form which is no longer capable of making singlet oxygen. Optimal systems of this invention are those providing maximum recycling of photoactivator.

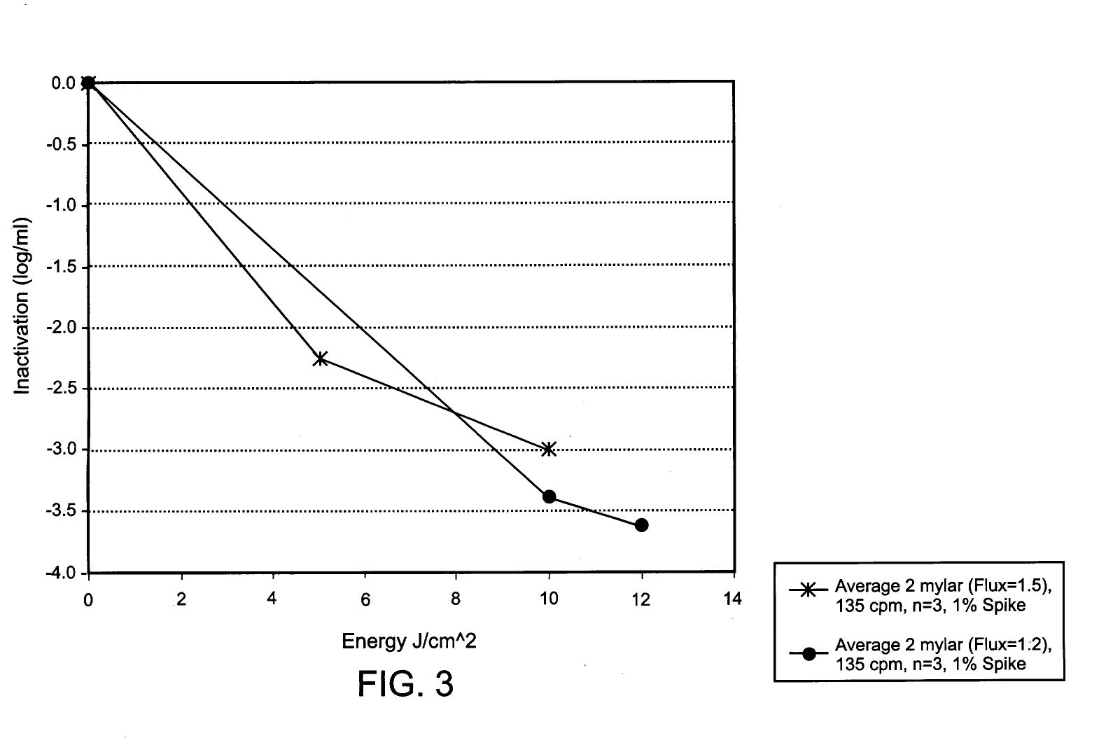

FIGS. 3 through 10 provide data for experiments (more fully explained in the Examples hereof) showing higher efficiency of pathogen inactivation using oxygen as compared to air; adjustment of light intensity to prevent premature exhaustion of photosensitizer and oxygen; use of mixing to provide enhanced dissolved oxygen to the fluid in an air atmosphere; and results of adding vitamin E to the fluid.

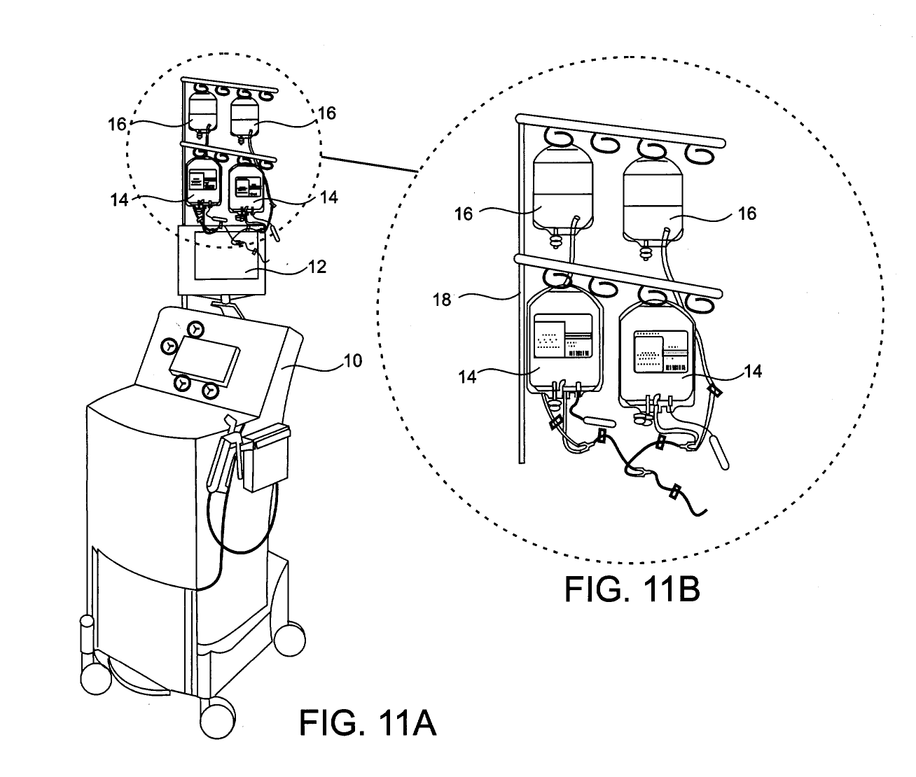

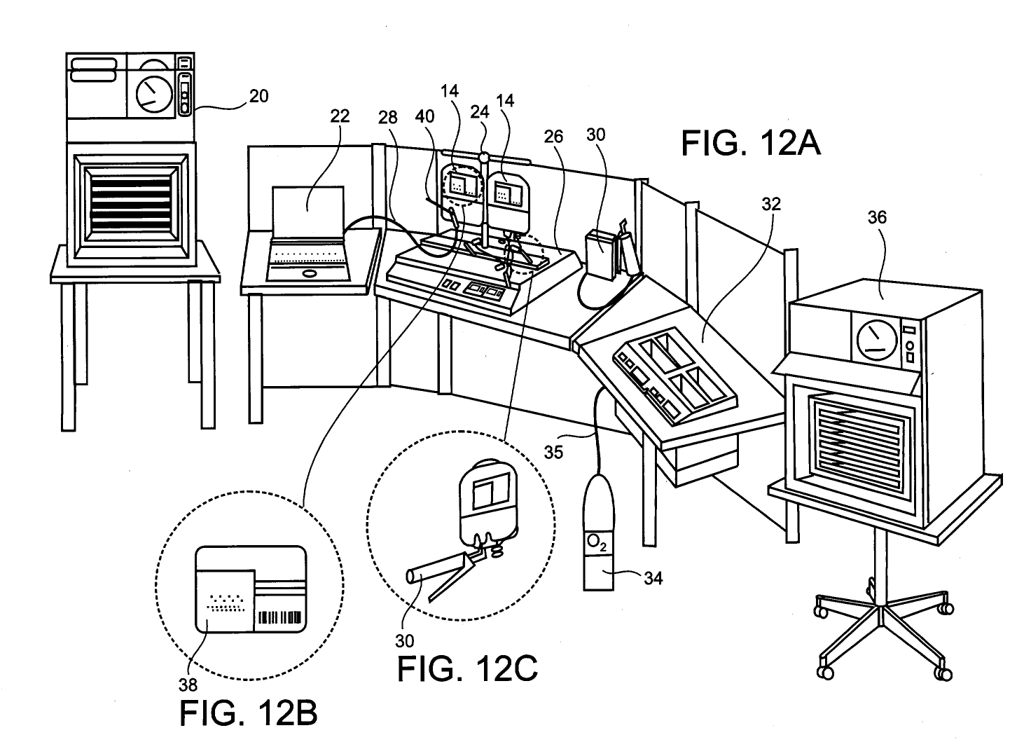

FIGS. 11 and 12 are described in relation to the collection of a double platelet product, however any type of blood product, either double or single, may be used in this invention. FIGS. 11A and 11B and 12A, 12B and 12C depict apparatuses used in a preferred embodiment of this invention for blood collection, aphersis, and pathogen eradication treatment. FIG. 11 shows a Trima apheresis apparatus manufactured by Gambro BCT (Lakewood, Colo.), however it should be noted that any type of apheresis apparatus may be used. As shown in FIG. 11A, the apheresis system includes apheresis apparatus 10 and touch screen 12 for controlling the process. Platelets may be collected from a donor into platelet collection bags 14 having a volume of about 600 ml each, and suspended from the bottom tier of a two-tier IV bar 18. Photoactivator bags 16 are suspended from the top tier of IV bar 18. In a preferred embodiment, the photosensitizer bags are two-compartment bags having a temporary seal between the compartments, e.g. as described in U.S. Patent Application No. 60/278,318, incorporated herein by reference to the extent not inconsistent herewith. One compartment may contain buffer and the other may contain photoactivator, preferably riboflavin. The two compartments are used to keep the components separate from each other during heat sterilization to prevent breakdown of the components, e.g. degradation of the riboflavin. After sterilization, the seal between the compartments is broken and the contents allowed to mix.

Each photosensitizer bag 16 is connected by tubing with the platelet collection bag 14 beneath it. The photosensitizer bags 16 contain a total volume of mixed photosensitizer and buffer of about 220 ml. The tubing connecting the photosensitizer and platelet collection bags preferably comprises a breakable barrier such as a frangible connector. Upon breaking the barrier, the contents of the photosensitizer bags are allowed to drain by gravity into the platelet collection bags.

After the photosensitizer bags are empty, they may be sealed off from the system using a radio frequency (RF) or heat sealing device, preferably an RF tubing sealer such as the Sebra Sealer (Sebra Engineering and Research Associates, Inc., Co., Phoenix, Ariz.). The empty photoactivator bags are discarded. One of the platelet collection bags is then elevated above the other and the contents of the upper bag allowed to drain into the lower bag.

Hyperconcentrated platelets are then collected in the lower collection bags 14 which contain the photosensitizer solution. After collection, the lower collection bags 14 are agitated to mix the contents to assure homogeneity of the platelet product. The upper and lower bags are then placed at the same elevation and allowed to roughly equilibrate via gravity. Each bag now contains approximately 80 ml of platelets and 220 ml of solution, at a platelet:solution ratio of about 27%:73%. The platelet collection bags are then sealed using bag sealer 30 (FIG. 12A) and transferred to the pathogen eradication treatment station.

The pathogen eradication treatment station is shown in FIGS. 12A, 12B, and 12C. The collected platelets in bags 14 containing photosensitizer/buffer solution are queued in a first blood bank shaker table 20 such as Helmer Model No. PC3200 (Helmer Co. Noblesville, Ind.). This process is detailed more fully in U.S. Pat. Nos. 6,258,577, 6227,337 and U.S. patent application Ser. No. 09/596,429, incorporated herein by reference to the extent not inconsistent herewith. The bags 14 are removed from the first Helmer apparatus 20 and their bar-coded labels 38 (FIG. 12B) are scanned with scanner 40 and the data from the scanner is sent to computer processor 22 via data cord 28. The bags 14 are then placed on pole holder 24 on scale 26 and weighed to assure that each bag contains the proper volume. This information is sent to the computer processor 22 and matched with the bar code on the bag label 38. The bags 14 are then taken to irradiator 32. Although irradiator 32 is shown as having slots through which the bags to be irradiated are inserted, it should be noted that other types of irradiators may also be used without departing from the spirit and scope of the invention. Other types of irradiators which may be used are described in U.S. Patent Applications 60/325,460 filed Sep. 27, 2001 and Ser. No. 09/962,029 filed Sep. 25, 2001, both of which are incorporated herein by reference to the extent not inconsistent herewith. An oxygen source 34 is connected to bags 14 by means of oxygen tubing 35 which comprises a sterile barrier such as a barrier filter to provide an oxygen atmosphere to each bag. Bags 14 are irradiated with visible light in irradiator 32 after oxygen has been added to the atmosphere therein. They may also be agitated by the irradiator. At the completion of irradiation, a signal is sent to computer processor 22 which is matched with the bar code on each bag 14 to show that the bag has received the decontamination treatment. Bags 14 are then placed in second Helmer apparatus 36.

### EXAMPLES

FIG. 3 shows inactivation of BVDV (as an analog virus for hepatitis C) as a function of energy using a solution comprising 27% platelets by volume in storage solution and 50 micromolar riboflavin, spiked with BVDV. A system involving a bank of Super Actinic 419 nm lights providing light in the visible spectrum was used to irradiate blood component bags having a fluid volume of 300 ml, and a gas volume of 150 ml. The fluid was placed in the bags with air and allowed to come to equilibrium. Irradiation was done while mixing the fluid using a linear mixer at 135 cpm. The lights were attenuated with two sheets of mylar to give a light flux of about 1.2 to about 1.5 J/cm²/min. In the experiments, in which data points are denoted by the asterisks and black dots, air was used for mixing into the fluid.

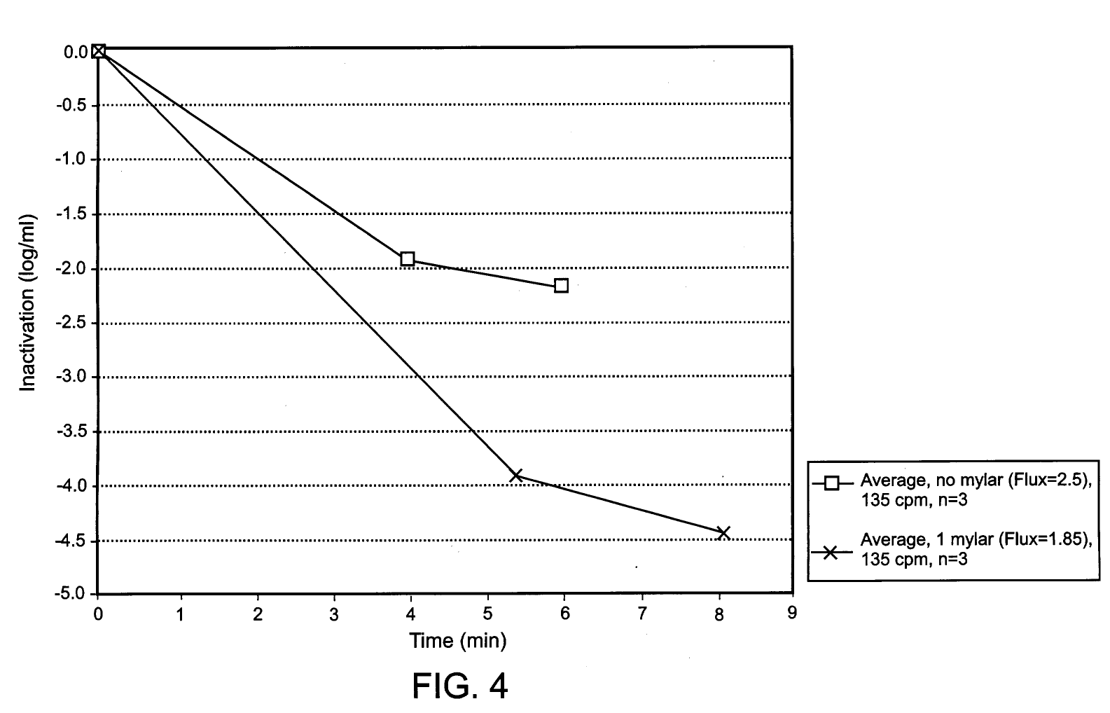

FIG. 4 shows BVDV inactivation as a function of time in minutes using a solution comprising 27% platelets by volume in storage solution containing 50 micromolar riboflavin, and 150 ml of air using bags as for FIG. 3 above. Mixing in all cases was done at 135 cpm. Light flux was adjusted in each experiment with no sheets of mylar in the first experiment (top line) giving a light flux of 2.5 J/cm²/min, 1 sheet of mylar (middle line) giving a light flux of 1.85 J/cm²/min, and two sheets of mylar (bottom line) giving a light flux of 1.5 J/cm²/min. In the presence of air, inactivation increased as light flux decreased. Too much energy favors irreversible conversion of the riboflavin to lumichrome which is not a photosensitizer under visible light, thus riboflavin is consumed and inactivation rate goes down.

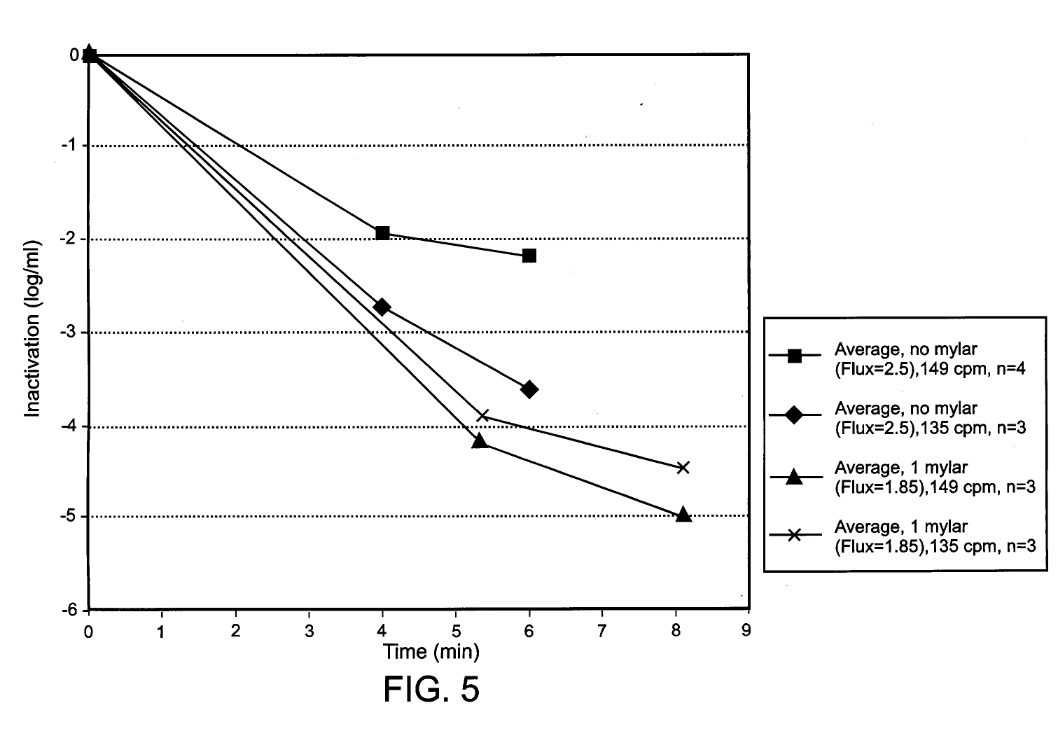

FIG. 5 is a graph showing inactivation of BVDV using a solution comprising 27% platelets by volume in storage solution containing 50 micromolar riboflavin, using bags as for FIG. 3 above. The bags were irradiated at light flux 2.5 J/cm²/min (no mylar placed over the light banks), and 1.85 J/cm²/min (one sheet of mylar placed over the light banks) while air was mixed into the fluids at mixing speeds of 135 and 149 cpm. When light flux was not adjusted with mylar, there was a large difference in pathogen inactivation depending on mixing speed. When one sheet of mylar was used to lower the light flux, oxygen and/or riboflavin were not consumed as quickly, and mixing speed was not as critical.

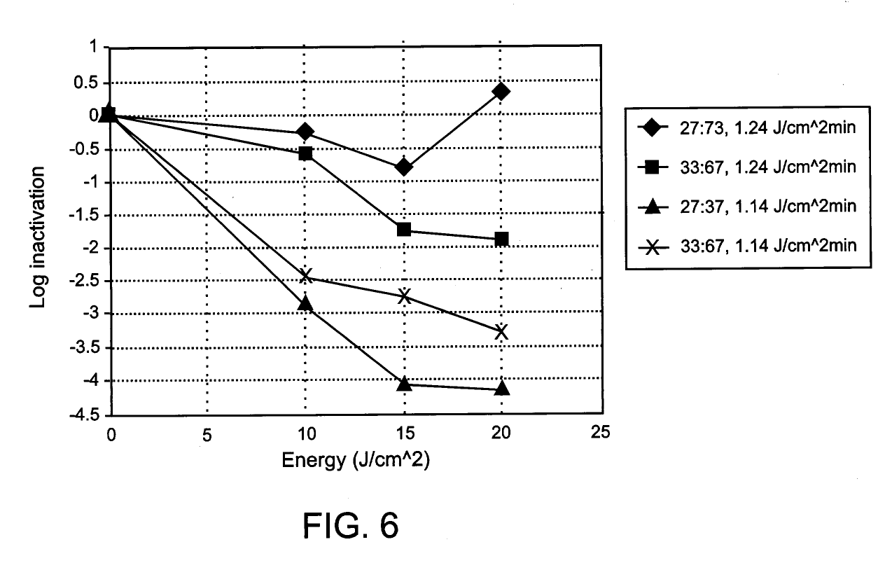

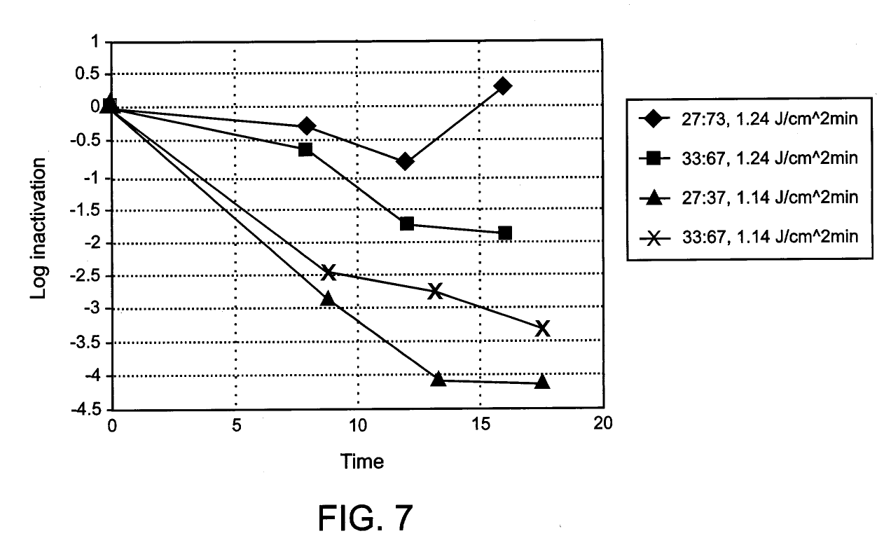

FIGS. 6 and 7 are graphs showing pseudorabies (PSR) virus inactivation as a function of energy (J/cm²/min) (FIG. 6) and time (FIG. 7) using 50 μm riboflavin, mixed with air at 149 cpm at 27:73 and 33:67 percent plasma carryover to percent storage solution at both 1.24 and 1.14 J/cm²/min achieved by placing two mylar sheets (for 1.24 J/cm²/min) or three mylar sheets for 1.14 J/cm²/min over the light banks. The lower light flux again gave more efficient inactivation per unit of energy delivered or per unit of time at both plasma carryover levels. The lower light flux gave faster and more complete inactivation at both plasma carryover levels.

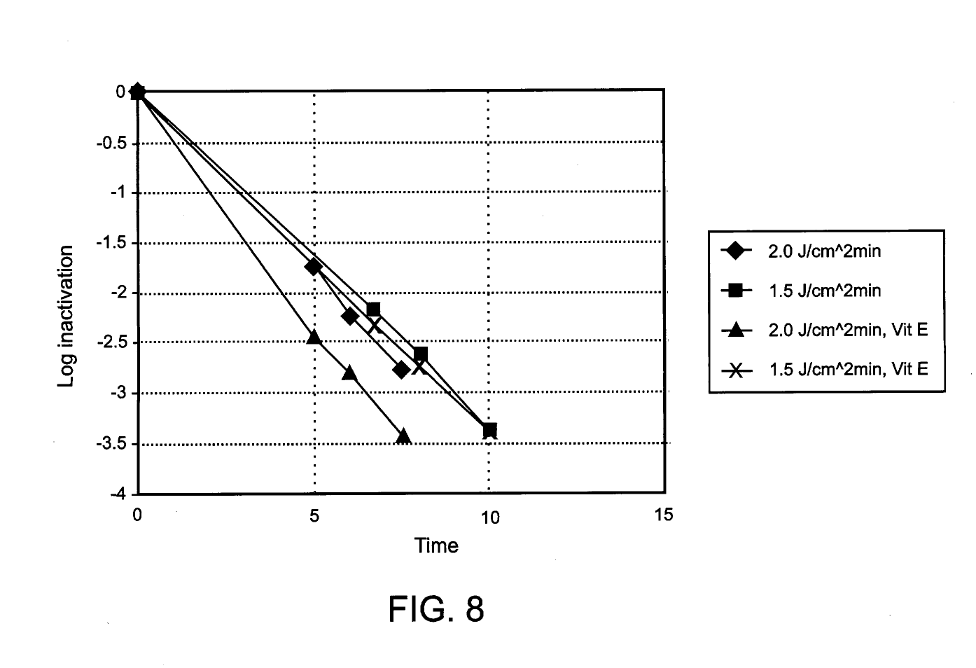

FIG. 8 is a graph showing PSR virus inactivation as a function of time (minutes) using 50 micromolar riboflavin, mixed with air at 149 cpm at 1.5 J/cm²/min with two sheets of mylar placed over the light banks and 2.0 J/cm²/min with one sheet of mylar placed over the light banks, with and without vitamin E. Vitamin E did not appear to interfere with pathogen inactivation, and in fact appeared to yield slightly better results in terms of rate of kill in the treatment condition using two sheets of mylar.

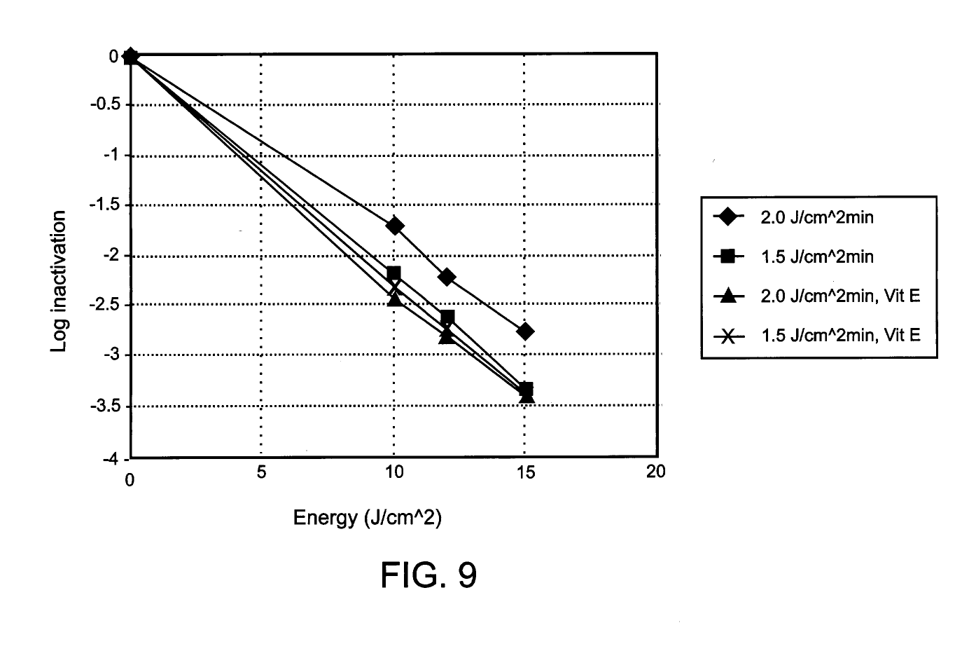

FIG. 9 is a graph showing PSR virus inactivation as a function of energy (J/cm²/min) using 50 micromolar riboflavin, mixed with air at 149 cpm at both 1.5 J/cm²/min using two sheets of mylar over the light banks, and 2.0 J/cm²/min with one sheet of mylar, with and without vitamin E. The least efficient kill per unit energy was achieved without vitamin E and using two sheets of mylar.

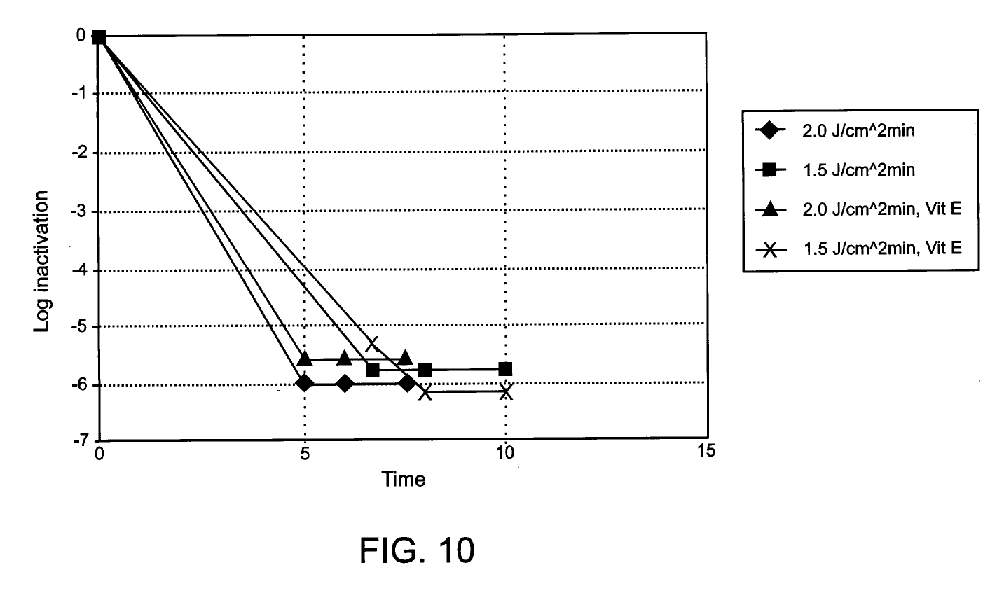

FIG. 10 is a graph showing BVDV inactivation as a function of time (minutes) using 50 micromolar riboflavin, mixed with air at 149 cpm at both 1.5 J/cm²/min using two sheets of mylar and 2.0 J/cm²/min using one sheet of mylar, with and without vitamin E. More complete pathogen inactivation was achieved with 1.5 J/cm²/min and vitamin E.

This invention has been illustrated using particular components, reagents and method steps. Equivalents known to the art can be substituted for any of these and are included within the scope of the following claims.

1. A method for treating a fluid to inactivate pathogens which may be present therein, comprising:
(a) mixing an inactivation-effective, substantially non-toxic amount of an endogenous photosensitizer or endogenously-based derivative photosensitizer with said fluid;
(b) increasing the dissolved oxygen content of said fluid to an amount sufficient to enhance reaction of the photosensitizer in which singlet oxygen and reactive oxygen species (ROS) are formed; and
(c) exposing said fluid to photoradiation of sufficient energy to activate the photosensitizer, for a sufficient time to substantially inactivate said pathogens.

2. The method of claim 1 wherein said photoradiation step comprises exposing said fluid to visible light energy.

3. The method of claim 1 wherein said fluid comprises one or more components selected from the group consisting of protein, blood and blood constituents.

4. The method of claim 3 wherein said fluid comprises a component selected from the group consisting of platelets, red cells, and plasma proteins.

5. The method of claim 4 wherein said fluid comprises platelets.

6. The method of claim 5 wherein said fluid comprises platelets in a solution comprising plasma and storage solution.

7. The method of claim 6 wherein the ratio of platelets to storage solution is between about 20:80 and about 90:10.

8. The method of claim 6 wherein the ratio of platelets to storage solution is between about 20:80 and about 35:65.

9. The method of claim 1 wherein said photosensitizer is selected from the group consisting of endogenous isoalloxazines and isoalloxazine derivative photosensitizers.

10. The method of claim 1 wherein said photosensitizer is selected from the group consisting of 7,8-dimethyl-10-ribityl isoalloxazine, 7,8-dimethylalloxazine, 7,8,10-trimethylisoalloxazine, alloxazine mononucleotide, and isoalloxazine-adenosine dinucleotide.

11. The method of claim 1 wherein said photosensitizer is an isoalloxazine derivative photo sensitizer.

12. The method of claim 1 wherein said photosensitizer is 7,8-dimethyl-10-ribityl isoalloxazine.

13. The method of claim 1 wherein said photosensitizer is present at a concentration between about 1 and about 200 micromolar.

14. The method of claim 1 wherein said photosensitizer concentrations is about 50 micromolar.

15. The method of claim 1 wherein said dissolved oxygen content of said fluid is increased to between about one and about five times the dissolved oxygen content that would be present in said fluid under an air atmosphere at ambient temperature and pressure without mixing.

16. The method of claim 1 wherein the dissolved oxygen content of said fluid is increased to about five times the dissolved oxygen content that would be present in said fluid under an air atmosphere at ambient temperature and pressure without mixing.

17. The method of claim 1 wherein the dissolved oxygen content of said fluid is increased by mixing said fluid with air.

18. The method of claim 17 wherein said mixing is performed by linearly mixing and shaking said fluid at a mixing speed between about 70 and about 150 cpm for sufficient time to equilibrate said fluid with said atmosphere.

19. The method of claim 18 wherein said mixing and shaking is performed for about five minutes.

20. The method of claim 1 wherein said dissolved oxygen content of said fluid is increased by placing said fluid in contact with an atmosphere of substantially pure oxygen for a period of time sufficient to increase said oxygen content.

21. The method of claim 1 wherein said dissolved oxygen content of said fluid is increased by mixing oxygen into said fluid.

22. The method of claim 21 wherein said mixing is performed at a mixing speed between about 70 and about 150 cpm for about one to about five minutes.

23. The method of claim 1 wherein agitation of said fluid is performed during irradiation.

24. The method of claim 23 wherein oxygen is added to said fluid during said irradiation.

25. The method of claim 1 wherein a lipophilic antioxidant is also added to said fluid in an amount effective to substantially prevent damage to platelets and/or red blood cells.

26. The method of claim 25 wherein said lipophilic antioxidant is selected from the group consisting of cysteine derivatives N-acetyl-L-cysteine, N-acetyl-D-cysteine (NAC), glutathione (GSH) L-cysteine; butylated hydroxyanisole (BHA), nordihydroguaiaretic acid (NDGA), dithiocarbamates, lipoic acid, and Vitamin E, vitamin E derivatives, dithiocarbamates, and alpha-lipoic acid.

27. The method of claim 25 wherein said lipophilic antioxidant is Vitamin E.

28. The method of claim 25 wherein said lipophilic antioxidant is added in an amount between about 0.25 mg/ml and about 2 mg/ml.

29. The method of claim 1 wherein the energy of said photoradiation is between about 5 and about 15 J/cm²/min.

30. The method of claim 1 wherein said photoradiation time is between about 2 and about 12 min.

31 The method of claim 1 wherein said pathogens are selected from the group consisting of extracellular and intracellular viruses, bacteria, bacteriophages, fungi, blood-transmitted parasites, and protozoa, and mixtures of any two or more of the foregoing.

32. The method of claim 31 wherein said viruses are selected from the group consisting of acquired immunodeficiency (HIV) virus, hepatitis A, B and C viruses, sindbis virus, cytomegalovirus, vesicular stomatitis virus, herpes simplex viruses, e.g. types I and II, human T-lymphotropic retroviruses, HTLV-III, lymphadenopathy virus LAV/IDAV, parvovirus, transfusion-transmitted (TT) virus, and Epstein-Barr virus, bovine viral diarrhea virus, pseudorabies, and mixtures of any two or more of the foregoing.

33. The method of claim 31 wherein said bacteriophages are selected from the group consisting of ΦX174, Φ6, λ, R₁₇, T₄, and T₂, and mixtures of any two or more of the foregoing.

34. The method of claim 31 wherein said bacteria are selected from the group consisting of P. aeruginosa, S. aureus, S. epidermidis, E. coli, K. pneumoniae, E. faecalis, B. subtilis, S. pneumoniae, S. pyrogenes, S. viridans, B. cereus, E. aerogenes, propionabacter, C. perfringes, E. cloacae, P. mirabilis, S. cholerasuis, S. liquifaciens, S. mitis, Y entercolitica, P. fluorescens, S. enteritidis, C. freundii, and S. marcescens, and mixtures of any two or more of the foregoing.

35. A blood product decontaminated by the method of claim 1.

36. A method for treating platelets to inactivate pathogens which may be present therein, comprising:
(a) mixing 7,8-dimethyl-10-ribityl isoalloxazine with a fluid comprising said platelets in storage solution at a ratio of about 27:73 platelets (in plasma):storage solution, whereby the 7,8-dimethyl-10-ribityl isoalloxazine concentration of said fluid is between about 1 and about 200 micromolar;
(b) increasing the dissolved oxygen content of said fluid to about five times that oxygen content of said fluid under an air atmosphere, by mixing air into said fluid, or by exposing said fluid to an atmosphere of substantially pure oxygen;
(c) exposing said fluid to photoradiation at an energy between about 10 and about 12 J/cm²/min to activate the photosensitizer, for at least about five to about seven minutes, to substantially inactivate said pathogens.

37. The method of claim 36 wherein said photoradiation step uses visible light.

38. The method of claim 36 also comprising adding vitamin E to said fluid.

39. A blood product decontaminated by the method of claim 36.

40. A biological composition comprising:
(a) a fluid;
(b) an inactivation-effective, substantially non-toxic amount of an endogenous photosensitizer or endogenously-based derivative photosensitizer;
(b) dissolved oxygen in an amount greater than would be present under an air atmosphere at ambient conditions without mixing.

41. The composition of claim 40 wherein said fluid comprises one or more components selected from the group consisting of red cells, platelets and plasma proteins.

42. The composition of claim 40 wherein said fluid comprises platelets.

43. The composition of claim 40 wherein said fluid comprises platelets and storage solution.

44. The composition of claim 43 wherein the ratio of platelets to storage solution is between about 20:80 and about 90:10.

45. The composition of claim 40 wherein said photosensitizer is selected from the group consisting of endogenous alloxazines.

46. The composition of claim 40 wherein said photosensitizer is an isoalloxazine derivative photosensitizer.

47. The composition of claim 40 wherein said photosensitizer is 7,8-dimethyl-10-ribityl isoalloxazine.

48. The composition of claim 40 wherein said photosensitizer is present at a concentration between about 1 and about 200 micromolar.

49. The composition of claim 40 wherein said photosensitizer concentration is about 50 micromolar.

50. The composition of claim 40 wherein said dissolved oxygen content of said fluid is between about one and about five times the dissolved oxygen content that would be present in said fluid under an air atmosphere at ambient temperature and pressure without mixing.

51. The composition of claim 40 wherein the dissolved oxygen content of said fluid is about five times the dissolved oxygen content that would be present in said fluid under an air atmosphere at ambient temperature and pressure without mixing.

52. The composition of claim 40 also comprising a lipophilic antioxidant.

53. The composition of claim 40 wherein said lipophilic antioxidant is selected from the group consisting of cysteine derivatives N-acetyl-L-cysteine, N-acetyl-D-cysteine (NAC), glutathione (GSH) L-cysteine; butylated hydroxyanisole (BHA), nordihydroguaiaretic acid (NDGA), dithiocarbamates, lipoic acid, and Vitamin E, vitamin E derivatives, dithiocarbamates, and alpha-lipoic acid.

54. The composition of claim 40 wherein said lipophilic antioxidant is Vitamin E.

55. The composition of claim 40 also comprising pathogens.

56. The composition of claim 55 wherein said pathogens are selected from the group consisting of extracellular and intracellular viruses, bacteria, bacteriophages, fungi, blood-transmitted parasites, and protozoa, and mixtures of any two or more of the foregoing.

57. The composition of claim 55 in which said pathogens have been substantially inactivated.

58. A blood component bag comprising the composition of claim 40.

59. A blood component bag comprising between about 100 and about 600 ml of the composition of claim 40.

60. The blood component bag of claim 58 also comprising air.

61. The blood component bag of claim 58 also containing a substantially pure oxygen atmosphere.

62. A decontamination system for a fluid comprising:
(a) a leak-proof transparent or translucent container for the fluid;
(b) a photosensitizer source for providing photosensitizer to said container, said photosensitizer source being connectible to an inlet of said container;
(c) an oxygen source connectible to an inlet of said container for providing oxygen to said container;
(d) a photoirradiator for irradiating said container;

63. The decontamination system of claim 62 also comprising an agitator for agitating said container.

64. The decontamination system of claim 62 also comprising a sterile barrier between said oxygen source and said container.

65. The decontamination system of claim 62 wherein said photosensitizer source contains photo sensitizer.

66. The decontamination system of claim 62 wherein said photosensitizer is an endogenous photosensitizer.

67. The decontamination system of claim 62 wherein said photosensitizer is selected from the group consisting of 7,8-dimethyl-10-ribityl isoalloxazine, 7,8-dimethylalloxazine, 7,8,10-trimethylisoalloxazine, alloxazine mononucleotide, isoalloxazine-adenosine dinucleotide, isoalloxazine derivative photosensitizers, and mixtures thereof.

68. The decontamination system of claim 62 wherein said photosensitizer is an isoalloxazine derivative.

69. The decontamination system of claim 62 wherein said photosensitizer is 7,8-dimethyl-10-ribityl isoalloxazine.

70. The decontamination system of claim 62 wherein said photosensitizer is present at a concentration between about 1 and about 200 micromolar.

71. The decontamination system of claim 62 wherein said container is a blood product collection bag.

72. The decontamination system of claim 71 wherein said blood product collection bag contains a blood product selected from the group consisting of platelets, red blood cells and plasma proteins.

73. The decontamination system of claim 62 wherein said blood product is platelets.

74. The decontamination system of claim 62 wherein said fluid comprises platelets (in plasma) and storage solution at a ratio between about 20:80 and about 90:10 platelets:storage solution.

75. The decontamination system of claim 62 wherein said irradiator is a visible light irradiator.

76. The decontamination system of claim 62 wherein said agitator is a linear mixer/shaker.

77. The decontamination system of claim 62 also comprising a scale for weighing said container.

78. The decontamination system of claim 62 also comprising a bar-coded label for said container and a scanner for reading said bar-coded label.

79. The decontamination system of claim 62 also comprising a computer processor for receiving, correlating and storing data comprising data identifying said container, the weight of said container, and the fact that said container has been irradiated.

80. The decontamination system of claim 62 also comprising at least one apparatus for maintaining temperature of said fluid and agitating said container before and/or after irradiation.

81. A method of making a decontamination system comprising:
(a) providing a set of components comprising:
(i) a leak-proof transparent or translucent container for the fluid;
(ii) a photosensitizer source for providing photosensitizer to said container, said photosensitizer source being connectible to an inlet of said container;
(iii) an oxygen source connectible to an inlet of said container for providing oxygen to said container;
(iv) a photoirradiator for irradiating said container; and
(b) assembling said components in proximity to each other.

82. The method of claim 81 also comprising connecting the photosensitizer source to the inlet of said container.

83. The method of claim 81 also comprising connecting the oxygen source to the inlet of said container.

84. A method of decontaminating a fluid comprising:
(a) providing a set of components comprising:
(i) a leak-proof transparent or translucent container for the fluid;
(ii) a photosensitizer source for providing photosensitizer to said container, said photosensitizer source being connectible to an inlet of said container;
(iii) an oxygen source connectible to an inlet of said container for providing oxygen to said container;
(iv) a photoirradiator for irradiating said container;
(b) assembling said components in proximity to each other;
(c) connecting said photosensitizer source to an inlet of said container;
(d) transferring photosensitizer from said photosensitizer source to said container;
(e) connecting said oxygen source to an inlet of said container;
(f) replacing the atmosphere in said container with oxygen from said oxygen source;
(g) positioning said container with respect to said irradiator such that radiation from said irradiator can reach fluid within said container;
(h) activating said irradiator to irradiate said fluid, thereby decontaminating said fluid.

85. The method of claim 84 also comprising providing an agitator to agitate said fluid within said container and activating said agitator to agitate said fluid.

86. The method of claim 85 wherein said agitation is performed simultaneously with irradiation of said container.

87. A method of increasing the storage life of photochemically decontaminated platelets platelets comprising:
(a) placing said platelets in a container larger than the volume of a solution containing said platelets; and
(b) dissolving an amount of oxygen in said solution greater than that would be dissolved in said solution under an air atmosphere at ambient conditions without agitation;
(c) adding a photoactivator to said solution and irradiating said solution to activate said photoactivator;
(d) removing oxygen from the atmosphere in said container; and
(e) storing said platelets.
